(19) United States
(12) Patent Application Publication
(10) Pub. No.: US 2024/0226048 A1
(43) Pub. Date: Jul. 11, 2024

(54) METHODS OF TREATMENT USING BEMPEDOIC ACID

(71) Applicant: Esperion Therapeutics, Inc., Ann Arbor, MI (US)

(72) Inventors: Leanne Tyndall Bloedon, Raleigh, NC (US); William J. Sasiela, Bloomingdale, NJ (US)

(21) Appl. No.: 18/296,628

(22) Filed: Apr. 6, 2023

Related U.S. Application Data
(60) Provisional application No. 63/479,105, filed on Jan. 9, 2023.

(51) Int. Cl. A61K 31/20; A61K 31/397; A61K 45/06; A61P 9/10

(52) U.S. Cl. CPC A61K 31/20; A61K 31/397; A61K 45/06; A61P 9/10; A61K 9/0053

(57) ABSTRACT

Provided herein are methods of reducing the risk of myocardial infarction or coronary revascularization in an adult with established cardiovascular disease or two or more risk factors for cardiovascular disease. Also provided herein are methods of reducing low-density lipoprotein cholesterol in an adult with primary hyperlipidemia. The methods described herein generally comprise administering to the adult an effective amount of bempedoic acid.

### CROSS-REFERENCE TO RELATED APPLICATIONS

This application claims priority to and the benefit of U.S. Provisional Patent Application No. 63/479,105, filed on Jan. 9, 2023, the entire disclosure of which is hereby incorporated by reference herein in its entirety.

### BACKGROUND

Administration of statins for elevated levels of low-density lipoprotein-cholesterol (LDL-C) represents the cornerstone of contemporary therapy to reduce the risk of major adverse cardiovascular events in both primary and secondary prevention patients. However, many patients report adverse musculoskeletal side effects that either prevents them from using statins or limits their ability to tolerate a dosage necessary to achieve their cholesterol targets. Withdrawal from or failure to maximize statin therapy are associated with increased risk of adverse cardiovascular events. Consequently, there is an unmet need to develop novel therapeutic strategies for reducing LDL-C and minimizing the risk of major adverse cardiovascular events in patients with elevated levels of LDL-C.

### SUMMARY

The present disclosure provides methods of using bempedoic acid to reduce the risk of myocardial infarction or coronary revascularization in an adult with established cardiovascular disease or two or more risk factors for cardiovascular disease. The disclosure additionally provides methods of using bempedoic acid to reduce low-density lipoprotein (LDL-C) in an adult with primary hyperlipidemia. The methods generally comprise administering to the adult a pharmaceutical formulation comprising an effective amount of bempedoic acid (e.g., 180 mg bempedoic acid).

In one aspect, the disclosure provides a method of reducing the risk of myocardial infarction in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof.

In another aspect, the disclosure provides a method of reducing the risk of coronary revascularization in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof.

In another aspect, the disclosure provides a method of reducing low-density lipoprotein cholesterol (LDL-C) in an adult with primary hyperlipidemia, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof, alone or in combination with another LDL-C-lowering therapy.

In another aspect, the disclosure provides a method of reducing low-density lipoprotein cholesterol (LDL-C) in an adult with primary hyperlipidemia, the method comprising orally administering daily to the adult a pharmaceutical formulation comprising 180 mg bempedoic acid; 10 mg ezetimibe; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof, alone or in combination with another LDL-C-lowering therapy.

### BRIEF DESCRIPTION OF FIGURES

FIG. 1A shows the percent change from baseline in low-density lipoprotein cholesterol (LDL-C) for the bempedoic acid and placebo treatment groups. The mean starting LDL-C for both groups was 139.0 mg/dL. To convert the values for cholesterol to millimoles per liter, multiply by 0.02586.

FIG. 1B shows the changes in high-sensitivity C-reactive protein (hsCRP) for the bempedoic acid and placebo treatment groups at timepoints during the trial. The median starting hsCRP was 2.3 mg/L.

FIG. 2A shows the cumulative incidence of the primary efficacy end point (a composite of death from coronary causes, nonfatal myocardial infarction, nonfatal stroke, or coronary revascularization). The insets show the same data on an enlarged y axis. The P values were calculated with the use of log-rank tests. MACE denotes major adverse cardiovascular events.

FIG. 2B shows the cumulative incidence of the key secondary endpoint (a composite of death from cardiovascular causes, nonfatal myocardial infarction, or nonfatal stroke). The insets show the same data on an enlarged y axis. The P values were calculated with the use of log-rank tests. MACE denotes major adverse cardiovascular events.

FIG. 2C shows the cumulative incidence of the key secondary end point (fatal and nonfatal myocardial infarction (MI)). The insets show the same data on an enlarged y axis. The P values were calculated with the use of log-rank tests.

FIG. 2D shows the cumulative incidence of the key secondary endpoint (coronary revascularization). The insets show the same data on an enlarged y axis. The P values were calculated with the use of log-rank tests.

### DETAILED DESCRIPTION

As generally described herein, the disclosure provides methods of reducing the risk of myocardial infarction or coronary revascularization in an adult with established cardiovascular disease or two or more risk factors for cardiovascular disease. The disclosure additionally provides methods of reducing low-density lipoprotein (LDL-C) in an adult with primary hyperlipidemia. The methods generally comprise administering to the adult a pharmaceutical formulation comprising an effective amount of bempedoic acid (e.g., 180 mg bempedoic acid).

### Definitions

To facilitate an understanding of the present disclosure, a number of terms and phrases are defined below.

Unless defined otherwise, all technical and scientific terms used herein have the same meaning as commonly understood by one of ordinary skill in the art to which this disclosure belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” refers to one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a +10%, +5%, +2%, or +1% variation from the nominal value unless otherwise indicated or inferred from the context.

It should be understood that the order of steps or order for performing certain actions is immaterial so long as the present disclosure remains operable. Moreover, two or more steps or actions may be conducted simultaneously.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a carrier, inert or active, making the composition especially suitable for diagnostic or therapeutic use in vivo or ex vivo.

The phrases “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, and general safety and purity standards as required by regulatory agencies that evaluate the safety and efficacy of pharmaceuticals and drug products, e.g., the U.S. Food and Drug Administration. “Pharmaceutically acceptable” and “pharmacologically acceptable” can mean approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present disclosure (e.g., bempedoic acid), which salt is compatible with pharmaceutical administration. For example, one or both of the carboxylic acid groups of bempedoic acid can be transformed to pharmaceutically acceptable salt(s).

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Nat, K⁺, Ca²⁺, NH₄⁺, and NW₄⁺ (where W can be a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “carrier” refers to a material, composition or vehicle, such as a liquid or solid filler, diluent, excipient, solvent or encapsulating material, involved in carrying or transporting a pharmaceutical agent such as bempedoic acid, or a pharmaceutically acceptable salt thereof, from one organ, or portion of the body, to another organ, or portion of the body.

As used herein, “pharmaceutically acceptable excipient” refers to a substance that aids the administration of an active agent to and/or absorption by a subject (e.g., an adult) and can be included in the compositions of the present disclosure without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. For examples of excipients, see Martin, Remington's Pharmaceutical Sciences, 15th Ed., Mack Publ. Co., Easton, PA (1975).

As used herein, “treat,” “treating” or “treatment” includes an action that occurs while a subject (e.g., an adult) is suffering from a specified disease, disorder or condition, which reduces the severity of the disease, disorder or condition, or retards or slows the progression of the disease, disorder or condition (e.g., lessening, reducing, modulating, ameliorating or eliminating, that results in the improvement of the condition, disease, disorder, and the like), or lessens, reduces, modulates, ameliorates or eliminates a symptom thereof. Treating can be curing, improving, or at least partially ameliorating the disorder. In certain embodiments, treating is curing the disease.

As used herein, “reducing” or “reduction” of a symptom or symptoms (and grammatical equivalents of this phrase) refers to decreasing of the severity or frequency of the symptom(s), or elimination of the symptom(s). For example, “reducing” or “reduction” of polycystic kidney disease may refer to a decrease in the rate of kidney cyst growth or a decrease in the rate of loss of kidney function.

As used herein, “reducing” or “reduction” of an elevated laboratory biomarker/parameter or vital sign associated with a disease disclosed herein may refer a decrease in the elevated laboratory biomarker/parameter or vital sign, for example, to a pre-determined clinically relevant endpoint (e.g., a clinically normal level).

As used herein, “subject” and “patient” are used interchangeably and refer to an organism to be treated by the methods and compositions of the present disclosure. Such organisms are preferably a mammal (e.g., human, mouse, rat, guinea pig, dog, cat, horse, cow, pig, or non-human primate, such as a monkey, chimpanzee, baboon, and rhesus), and more preferably, a human. In certain embodiments, the subject is an adult human.

As used herein, “solid dosage form” refers to a pharmaceutical dose(s) in solid form, e.g., tablets, capsules, granules, powders, sachets, reconstitutable powders, dry powder inhalers and chewables.

As used herein, “immediate release” refers to a dosage form that has not been engineered to modify or control the release of the active ingredient.

As used herein, “sustained release” refers to a dosage form designed to release a drug at a predetermined (but not necessarily constant) rate in order to maintain a desired range of drug concentration over a specific period of time, e.g., 8 hours, 12 hours, 16 hours, 20 hours, 24 hours, etc., with minimum side effects.

As used herein, “fixed-dose combination” refers to a form in which the active ingredients (e.g., bempedoic acid and ezetimibe) are both administered to a patient simultaneously in the form of a single entity or dosage.

As used herein, “administering” refers to oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject (e.g., an adult). Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., bempedoic acid and ezetimibe). Bempedoic acid, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, “disease,” “disorder,” “condition,” or “illness,” which can be used interchangeably herein unless otherwise understood from the context, refers to a state of being or health status of a patient or subject (e.g., an adult) capable of being treated with a compound, pharmaceutical material, pharmaceutical composition, or method provided herein.

As used herein, “effective amount” or “therapeutically-effective amount” refers to the amount of a compound (e.g., bempedoic acid), a combination of compounds (e.g., bempedoic acid and ezetimibe), a pharmaceutical composition (e.g., a pharmaceutical composition of the present disclosure), or a fixed-dose combination (e.g., a fixed-dose combination of the present disclosure) sufficient to effect beneficial or desired results. An effective amount can be administered in one or more administrations, applications or dosages and is not intended to be limited to a particular formulation or administration route.

### Bempedoic Acid

Bempedoic acid is a non-statin drug indicated as an adjunct to diet and maximally tolerated statin therapy for the treatment of adults with heterozygous familial hypercholesterolemia or established atherosclerotic cardiovascular disease who require additional lowering of LDL-C. It functions through inhibition of adenosine triphosphate-citrate lyase (ACL). It behaves as a prodrug in vivo, where it is converted to the active species bempedoic acid-CoA by endogenous liver acyl-Coenzyme (CoA) synthetase (ACS) activity. A specific ACS isozyme, very long-chain acyl-CoA synthetase (ACSVL1), is required to form the active species. Bempedoic acid can also activate the metabolic sensor AMP-activated protein kinase (AMPK).

Bempedoic acid may also be represented by the structure of Formula (I):

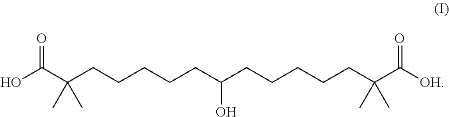 [SMILES: CC(C)(CCCCCC(O)CCCCCC(C)(C)C(=O)O)C(=O)O]

Bempedoic acid and a process for synthesizing it are disclosed in the U.S. Pat. Nos. 7,335,799 and 11,407,705; and International Publication No. WO 2020/257571 A1, each of which is herein incorporated by reference. Bempedoic acid may also be referred to as ETC-1002, ESP-55016, or under the tradenames Nexletol® and Nilemdo®.

In various embodiments, bempedoic acid may be used for the treatment and/or prevention of a variety of conditions, diseases and disorders described herein. The methods of treating a condition, disease, or disorder described herein generally comprise administering to an adult in need thereof, a therapeutically effective amount of bempedoic acid to treat the condition, disease, or disorder.

In certain embodiments, bempedoic acid may be used for reducing the risk of myocardial infarction in an adult with established cardiovascular disease or two or more risk factors for cardiovascular disease.

In certain embodiments, bempedoic acid may be used for reducing the risk of coronary revascularization in an adult with established cardiovascular disease or two or more risk factors for cardiovascular disease.

In certain embodiments, bempedoic acid may be used for reducing low-density lipoprotein cholesterol (LDL-C) in an adult with primary hyperlipidemia.

In certain embodiments, the fixed-dose combinations and pharmaceutical formulations disclosed herein comprise a crystalline form of bempedoic acid. As used herein, “crystalline form of bempedoic acid” may refer to a crystalline form of the free acid form of bempedoic acid or a crystalline form of a pharmaceutically acceptable salt of bempedoic acid. See, e.g., International Publication No. WO 2020/257573 A1, which is herein incorporated by reference.

In certain embodiments, the fixed-dose combinations and pharmaceutical formulations disclosed herein comprise a high purity crystalline form of bempedoic acid.

In certain embodiments, the fixed-dose combinations and pharmaceutical formulations disclosed herein comprise a pharmaceutical material comprising bempedoic acid.

In various embodiments, a pharmaceutical material generally comprises a crystalline form of bempedoic acid, wherein the pharmaceutical material comprises bempedoic acid, or a pharmaceutically acceptable salt thereof, in an amount greater than 99.0% by weight based on the total weight of the pharmaceutical material. In some embodiments, the amount of bempedoic acid in the pharmaceutical material is greater than about 99.1%, greater than about 99.2%, greater than about 99.3%, greater than about 99.4%, greater than about 99.5%, greater than about 99.6%, greater than about 99.7%, greater than about 99.8%, greater than about 99.85%, greater than about 99.9%, greater than about 99.95%, or greater than about 99.98% by weight of the total weight of the pharmaceutical material. In some embodiments, the pharmaceutical material comprises bempedoic acid in an amount greater than 99.5% by weight based on the total weight of the pharmaceutical material. In some embodiments, the pharmaceutical material comprises bempedoic acid in an amount greater than 99.7% by weight based on the total weight of the pharmaceutical material. In some embodiments, the pharmaceutical material comprises bempedoic acid in an amount greater than 99.9% by weight based on the total weight of the pharmaceutical material.

In various embodiments, a pharmaceutical material generally comprises a crystalline form of bempedoic acid, wherein the pharmaceutical material comprises bempedoic acid in an amount greater than 99.0% by weight based on the total weight of the pharmaceutical material. In some embodiments, the amount of bempedoic acid in the pharmaceutical material is greater than about 99.1%, greater than about 99.2%, greater than about 99.3%, greater than about 99.4%, greater than about 99.5%, greater than about 99.6%, greater than about 99.7%, greater than about 99.8%, greater than about 99.85%, greater than about 99.9%, greater than about 99.95%, or greater than about 99.98% by weight of the total weight of the pharmaceutical material. In some embodiments, the pharmaceutical material comprises bempedoic acid in an amount greater than 99.5% by weight based on the total weight of the pharmaceutical material. In some embodiments, the pharmaceutical material comprises bempedoic acid in an amount greater than 99.7% by weight based on the total weight of the pharmaceutical material. In some embodiments, the pharmaceutical material comprises bempedoic acid in an amount greater than 99.9% by weight based on the total weight of the pharmaceutical material.

In certain embodiments, the pharmaceutical material comprises bempedoic acid in an amount of from about 98% to about 102% by weight based on the total weight of the pharmaceutical material (anhydrous, solvent-free basis), as determined by a high-performance liquid chromatography (HPLC) assay.

In certain embodiments, the HPLC assay comprises one or more of:
(i) a Waters XBridge BEH C18 column (4.6 mm i.d.×150 mm, 2.5 pm);
(ii) a column temperature of about 40° C.;
(iii) a mobile phase comprising about 0.05% phosphoric acid in water/acetonitrile (about 50:50);
(iv) isocratic elution;
(v) a flow rate of about 1.2 mL/minute;
(vi) a sample temperature of ambient temperature;
(vii) detection at 215 nm; and
(viii) the retention time of the compound of formula (I) is about 4.6 minutes. In some embodiments, the HPLC assay comprises each of the above, i.e., (i)-(viii).

In certain embodiments, the crystalline form of bempedoic acid may be a crystalline form of bempedoic acid as characterized in International Publication Nos. WO 2020/257571 A1 and WO 2020/257573 A1, each of which is herein incorporated by reference. A crystalline form of bempedoic acid may be characterized, for example, by an X-ray powder diffraction pattern or peak(s), and/or other characteristic properties such as melting point and hygroscopicity. Crystalline forms of bempedoic acid may include, but are not limited to, cocrystals (e.g., an aspartame cocrystal and a palmitic acid cocrystal), crystalline salts (e.g., an ammonium salt, a sodium salt, a potassium salt, a calcium salt, a lysine salt, a diethylamine salt, an ethylenediamine salt, a piperazine salt, a betaine salt, a tromethamine salt, and an isonicotinamide salt).

### Ezetimibe

Ezetimibe is a cholesterol absorption inhibitor indicated to reduce total cholesterol, low-density lipoprotein (LDL), apolipoprotein B (apo B), and non-high-density lipoprotein (HDL) in patients with primary hyperlipidemia, mixed hyperlipidemia, familial hypercholesterolemia (FH), and homozygous sitosterolemia (phytosterolemia).

Ezetimibe may be used in the fixed-dose combinations, pharmaceutical compositions, and methods of treatment described herein. In various embodiments, the fixed-dose combinations and pharmaceutical compositions provided herein comprise ezetimibe. Ezetimibe is represented by the structure of Formula (II):

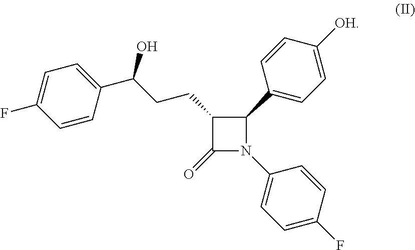 [SMILES: O=C1[C@H](CC[C@H](O)c2ccc(F)cc2)[C@@H](c2ccc(O)cc2)N1c1ccc(F)cc1]

Ezetimibe and its process of manufacture are disclosed in, for example, U.S. Pat. No. 5,631,365, which is incorporated herein by reference. Ezetimibe may be administered as an oral dosage form (e.g., a tablet). Ezetimibe may also be referred to as (3R,4S)-1-(4-fluorophenyl)-3-[(3S)-3-(4-fluorophenyl)-3-hydroxypropyl]-4-(4-hydroxyphenyl)azetidin-2-one, or under the tradenames Zetia® and Ezetrol®.

In various embodiments, ezetimibe may be used for the treatment or prevention of a variety of conditions, diseases, and disorders described herein. The methods of preventing or treating a condition, disease, or disorder described herein generally comprise administering to an adult a therapeutically effective amount of ezetimibe to prevent or treat the condition, disease, or disorder.

In certain embodiments, ezetimibe may be used in combination with bempedoic acid for reducing low-density lipoprotein cholesterol (LDL-C) in an adult with primary hyperlipidemia.

### Fixed-Dose Combinations

Disclosed herein are fixed-dose combinations generally comprising bempedoic acid and ezetimibe. A fixed-dose combination of bempedoic acid and ezetimibe may also be referred to herein under the tradenames Nexlizet® and Nustendi®.

In various embodiments, a fixed-dose combination described herein is a solid dosage form comprising bempedoic acid and ezetimibe.

In various embodiments, a fixed-dose combination described herein is a solid dosage form comprising a pharmaceutical formulation described herein.

In certain embodiments, the fixed-dose combination comprises about 180 mg bempedoic acid. In some embodiments, the fixed-dose combination comprises 180 mg bempedoic acid.

In some embodiments, the fixed-dose combination comprises about 10 mg ezetimibe. In some embodiments, the fixed-dose combination comprises 10 mg ezetimibe.

In certain embodiments, the fixed-dose combination comprises about 180 mg bempedoic acid and about 10 mg ezetimibe. In some embodiments, the fixed-dose combination comprises 180 mg bempedoic acid and 10 mg ezetimibe.

In some embodiments, the fixed-dose combinations disclosed herein are formulated for oral delivery. In some embodiments, the fixed-dose combinations disclosed herein are formulated as an oral dosage form. Examples of oral dosage forms include, but are not limited to, a drench, a tablet, a capsule, a softgel capsule, a cachet, a pill, an emulsion, a lozenge, a solution, a suspension, a bolus, a powder, an elixir or syrup, a pastille, a mouthwash, a granule, or a paste for application to the tongue. In some embodiments, the fixed-dose combination is formulated as a tablet.

In certain embodiments, the fixed-dose combinations provided herein can be used for the treatment of a disease, disorder, or condition described herein.

In certain embodiments, a fixed-dose combination described herein may be used for reducing the risk of myocardial infarction in an adult with established cardiovascular disease or two or more risk factors for cardiovascular disease.

In certain embodiments, a fixed-dose combination described herein may be used for reducing the risk of coronary revascularization in an adult with established cardiovascular disease or two or more risk factors for cardiovascular disease.

In certain embodiments, a fixed-dose combination described herein may be used for reducing low-density lipoprotein cholesterol (LDL-C) in an adult with primary hyperlipidemia.

### Pharmaceutical Formulations

Also disclosed herein are pharmaceutical formulations generally comprising bempedoic acid, and one or more pharmaceutically acceptable excipients.

In certain embodiments, the one or more pharmaceutically acceptable excipients is selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof.

In various embodiments, provided herein is a pharmaceutical formulation comprising bempedoic acid; colloidal silicon dioxide; hydroxyl propyl cellulose; lactose monohydrate; magnesium stearate; microcrystalline cellulose; and sodium starch glycolate.

In various embodiments, provided herein is a pharmaceutical formulation comprising bempedoic acid; colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the pharmaceutical formulation further comprises ezetimibe.

In various embodiments, provided herein is a pharmaceutical formulation comprising bempedoic acid; ezetimibe; colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the amount of bempedoic acid in a pharmaceutical formulation described herein is about 45% (w/w) to about 55% (w/w). In certain embodiments, the amount of bempedoic acid in a pharmaceutical formulation described herein is about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), or about 55% (w/w). In certain embodiments, the amount of bempedoic acid in a pharmaceutical formulation described herein is about 47% (w/w). In certain embodiments, the amount of bempedoic acid in a pharmaceutical formulation described herein is about 48% (w/w). In certain embodiments, the amount of bempedoic acid in a pharmaceutical formulation described herein is about 49% (w/w). In certain embodiments, the amount of bempedoic acid in a pharmaceutical formulation described herein is about 50% (w/w). In certain embodiments, the amount of bempedoic acid in a pharmaceutical formulation described herein is about 51% (w/w). In certain embodiments, the amount of bempedoic acid in a pharmaceutical formulation described herein is about 52% (w/w).

In certain embodiments, the amount of bempedoic acid in a pharmaceutical formulation described herein is 47% (w/w), 47.1% (w/w), 47.2% (w/w), 47.3% (w/w), 47.4% (w/w), 47.5% (w/w), 47.6% (w/w), 47.7% (w/w), 47.8% (w/w), 47.9% (w/w), 48% (w/w), 48.1% (w/w), 48.2% (w/w), 48.3% (w/w), 48.4% (w/w), 48.5% (w/w), 48.6% (w/w), 48.7% (w/w), 48.8% (w/w), 48.9% (w/w), 49% (w/w), 49.1% (w/w), 49.2% (w/w), 49.3% (w/w), 49.4% (w/w), 49.5% (w/w), 49.6% (w/w), 49.7% (w/w), 49.8% (w/w), 49.9% (w/w), 50% (w/w), 50.1% (w/w), 50.2% (w/w), 50.3% (w/w), 50.4% (w/w), 50.5% (w/w), 50.6% (w/w), 50.7% (w/w), 50.8% (w/w), 50.9% (w/w), 51% (w/w), 51.1% (w/w), 51.2% (w/w), 51.3% (w/w), 51.4% (w/w), 51.5% (w/w), 51.6% (w/w), 51.7% (w/w), 51.8% (w/w), 51.9% (w/w), or 52% (w/w).

In certain embodiments, the amount of bempedoic acid in a pharmaceutical formulation described herein is about 180 mg. In certain embodiments, the amount of bempedoic acid in a pharmaceutical formulation described herein is 180 mg.

In certain embodiments, the amount of ezetimibe in a pharmaceutical formulation described herein is 0% (w/w) to about 3% (w/w). In certain embodiments, the amount of ezetimibe in a pharmaceutical formulation described herein is about 2.5% (w/w). In certain embodiments, the amount of ezetimibe in a pharmaceutical formulation described herein is about 2.6% (w/w). In certain embodiments, the amount of ezetimibe in a pharmaceutical formulation described herein is about 2.7% (w/w). In certain embodiments, the amount of ezetimibe in a pharmaceutical formulation described herein is about 2.8% (w/w). In certain embodiments, the amount of ezetimibe in a pharmaceutical formulation described herein is about 2.9% (w/w). In certain embodiments, the amount of ezetimibe in a pharmaceutical formulation described herein is about 3.0% (w/w).

In certain embodiments, the amount of ezetimibe in a pharmaceutical formulation described herein is about 10 mg. In certain embodiments, the amount of ezetimibe in a pharmaceutical formulation described herein is 10 mg.

In certain embodiments, the amount of colloidal silicon dioxide in a pharmaceutical formulation described herein is about 1% (w/w) to about 2% (w/w). In certain embodiments, the amount of colloidal silicon dioxide in a pharmaceutical formulation described herein is about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), or about 2% (w/w). In certain embodiments, the amount of colloidal silicon dioxide in a pharmaceutical formulation described herein is about 1% (w/w). In certain embodiments, the amount of colloidal silicon dioxide in a pharmaceutical formulation described herein is about 1.1% (w/w). In certain embodiments, the amount of colloidal silicon dioxide in a pharmaceutical formulation described herein is about 1.2% (w/w). In certain embodiments, the amount of colloidal silicon dioxide in a pharmaceutical formulation described herein is about 1.3% (w/w). In certain embodiments, the amount of colloidal silicon dioxide in a pharmaceutical formulation described herein is about 1.4% (w/w). In certain embodiments, the amount of colloidal silicon dioxide in a pharmaceutical formulation described herein is about 1.5% (w/w).

In certain embodiments, the amount of colloidal silicon dioxide in a pharmaceutical formulation described herein is about 3 mg to about 6 mg. In certain embodiments, the amount of colloidal silicon dioxide in a pharmaceutical formulation described herein is about 3 mg, about 3.5 mg, about 4 mg, about 4.5 mg, about 5 mg, about 5.5 mg, or about 6 mg. In certain embodiments, the amount of colloidal silicon dioxide in a pharmaceutical formulation described herein is about 3 mg. In certain embodiments, the amount of colloidal silicon dioxide in a pharmaceutical formulation described herein is about 3.5 mg. In certain embodiments, the amount of colloidal silicon dioxide in a pharmaceutical formulation described herein is about 4 mg. In certain embodiments, the amount of colloidal silicon dioxide in a pharmaceutical formulation described herein is about 4.5 mg. In certain embodiments, the amount of colloidal silicon dioxide in a pharmaceutical formulation described herein is about 5 mg. In certain embodiments, the amount of colloidal silicon dioxide in a pharmaceutical formulation described herein is about 5.5 mg. In certain embodiments, the amount of colloidal silicon dioxide in a pharmaceutical formulation described herein is about 6 mg.

In certain embodiments, the amount of the hydroxyl propyl cellulose in a pharmaceutical formulation described herein is about 3% (w/w) to about 5% (w/w). In certain embodiments, the amount of the hydroxyl propyl cellulose in a pharmaceutical formulation described herein is about 3% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), or about 5% (w/w). In certain embodiments, the amount of the hydroxyl propyl cellulose in a pharmaceutical formulation described herein is about 3% (w/w). In certain embodiments, the amount of the hydroxyl propyl cellulose in a pharmaceutical formulation described herein is about 3.1% (w/w). In certain embodiments, the amount of the hydroxyl propyl cellulose in a pharmaceutical formulation described herein is about 3.2% (w/w). In certain embodiments, the amount of the hydroxyl propyl cellulose in a pharmaceutical formulation described herein is about 3.3% (w/w). In certain embodiments, the amount of the hydroxyl propyl cellulose in a pharmaceutical formulation described herein is about 3.4% (w/w). In certain embodiments, the amount of the hydroxyl propyl cellulose in a pharmaceutical formulation described herein is about 3.5% (w/w). In certain embodiments, the amount of the hydroxyl propyl cellulose in a pharmaceutical formulation described herein is about 4.6% (w/w). In certain embodiments, the amount of the hydroxyl propyl cellulose in a pharmaceutical formulation described herein is about 4.7% (w/w). In certain embodiments, the amount of the hydroxyl propyl cellulose in a pharmaceutical formulation described herein is about 4.8% (w/w). In certain embodiments, the amount of the hydroxyl propyl cellulose in a pharmaceutical formulation described herein is about 4.9% (w/w). In certain embodiments, the amount of the hydroxyl propyl cellulose in a pharmaceutical formulation described herein is about 5.0% (w/w).

In certain embodiments, the amount of the hydroxyl propyl cellulose in a pharmaceutical formulation described herein is about 10 mg to about 20 mg. In certain embodiments, the amount of the hydroxyl propyl cellulose in a pharmaceutical formulation described herein is about 10 mg, about 11 mg, about 12 mg, about 13 mg, about 14 mg, about 15 mg, about 16 mg, about 17 mg, about 18 mg, about 19 mg, or about 20 mg. In certain embodiments, the amount of the hydroxyl propyl cellulose in a pharmaceutical formulation described herein is about 10 mg. In certain embodiments, the amount of the hydroxyl propyl cellulose in a pharmaceutical formulation described herein is about 11 mg. In certain embodiments, the amount of the hydroxyl propyl cellulose in a pharmaceutical formulation described herein is about 12 mg. In certain embodiments, the amount of the hydroxyl propyl cellulose in a pharmaceutical formulation described herein is about 13 mg. In certain embodiments, the amount of the hydroxyl propyl cellulose in a pharmaceutical formulation described herein is about 14 mg. In certain embodiments, the amount of the hydroxyl propyl cellulose in a pharmaceutical formulation described herein is about 15 mg. In certain embodiments, the amount of the hydroxyl propyl cellulose in a pharmaceutical formulation described herein is about 16 mg.

In certain embodiments, the amount of lactose monohydrate in a pharmaceutical formulation described herein is about 8% (w/w) to about 20% (w/w). In certain embodiments, the amount of lactose monohydrate in a pharmaceutical formulation described herein is about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), or about 20% (w/w). In certain embodiments, the amount of lactose monohydrate in a pharmaceutical formulation described herein is about 8% (w/w). In certain embodiments, the amount of lactose monohydrate in a pharmaceutical formulation described herein is about 9% (w/w). In certain embodiments, the amount of lactose monohydrate in a pharmaceutical formulation described herein is about 10% (w/w). In certain embodiments, the amount of lactose monohydrate in a pharmaceutical formulation described herein is about 11% (w/w). In certain embodiments, the amount of lactose monohydrate in a pharmaceutical formulation described herein is about 17% (w/w). In certain embodiments, the amount of lactose monohydrate in a pharmaceutical formulation described herein is about 18% (w/w). In certain embodiments, the amount of lactose monohydrate in a pharmaceutical formulation described herein is about 19% (w/w). In certain embodiments, the amount of lactose monohydrate in a pharmaceutical formulation described herein is about 20% (w/w).

In certain embodiments, the amount of the hydroxyl propyl cellulose in a pharmaceutical formulation described herein is about 20 mg to about 80 mg. In certain embodiments, the amount of the hydroxyl propyl cellulose in a pharmaceutical formulation described herein is about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg. In certain embodiments, the amount of the hydroxyl propyl cellulose in a pharmaceutical formulation described herein is about 20 mg. In certain embodiments, the amount of the hydroxyl propyl cellulose in a pharmaceutical formulation described herein is about 25 mg. In certain embodiments, the amount of the hydroxyl propyl cellulose in a pharmaceutical formulation described herein is about 30 mg. In certain embodiments, the amount of the hydroxyl propyl cellulose in a pharmaceutical formulation described herein is about 35 mg. In certain embodiments, the amount of the hydroxyl propyl cellulose in a pharmaceutical formulation described herein is about 65 mg. In certain embodiments, the amount of the hydroxyl propyl cellulose in a pharmaceutical formulation described herein is about 70 mg. In certain embodiments, the amount of the hydroxyl propyl cellulose in a pharmaceutical formulation described herein is about 75 mg. In certain embodiments, the amount of the hydroxyl propyl cellulose in a pharmaceutical formulation described herein is about 80 mg.

In certain embodiments, the amount of magnesium stearate in a pharmaceutical formulation described herein is about 1% (w/w) to about 3% (w/w). In certain embodiments, the amount of magnesium stearate in a pharmaceutical formulation described herein is about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), or about 3% (w/w). In certain embodiments, the amount of magnesium stearate in a pharmaceutical formulation described herein is about 1% (w/w). In certain embodiments, the amount of magnesium stearate in a pharmaceutical formulation described herein is about 1.1% (w/w). In certain embodiments, the amount of magnesium stearate in a pharmaceutical formulation described herein is about 1.2% (w/w). In certain embodiments, the amount of magnesium stearate in a pharmaceutical formulation described herein is about 1.3% (w/w). In certain embodiments, the amount of magnesium stearate in a pharmaceutical formulation described herein is about 2.7% (w/w). In certain embodiments, the amount of magnesium stearate in a pharmaceutical formulation described herein is about 2.8% (w/w). In certain embodiments, the amount of magnesium stearate in a pharmaceutical formulation described herein is about 2.9% (w/w). In certain embodiments, the amount of magnesium stearate in a pharmaceutical formulation described herein is about 3% (w/w).

In certain embodiments, the amount of the magnesium stearate in a pharmaceutical formulation described herein is about 2 mg to about 10 mg. In certain embodiments, the amount of the magnesium stearate in a pharmaceutical formulation described herein is about 2 mg, about 3 mg, about 4 mg, about 5 mg, about 6 mg, about 7 mg, about 8 mg, about 9 mg, or about 10 mg. In certain embodiments, the amount of the magnesium stearate in a pharmaceutical formulation described herein is about 3 mg. In certain embodiments, the amount of the magnesium stearate in a pharmaceutical formulation described herein is about 4 mg. In certain embodiments, the amount of the magnesium stearate in a pharmaceutical formulation described herein is about 5 mg. In certain embodiments, the amount of the magnesium stearate in a pharmaceutical formulation described herein is about 6 mg. In certain embodiments, the amount of the magnesium stearate in a pharmaceutical formulation described herein is about 7 mg. In certain embodiments, the amount of the magnesium stearate in a pharmaceutical formulation described herein is about 8 mg. In certain embodiments, the amount of the magnesium stearate in a pharmaceutical formulation described herein is about 9 mg.

In certain embodiments, the amount of the microcrystalline cellulose in a pharmaceutical formulation described herein is about 15% (w/w) to about 20% (w/w). In certain embodiments, the amount of the microcrystalline cellulose in a pharmaceutical formulation described herein is about 15% (w/w), about 16% (w/w), about 17% (w/w), about 18% (w/w), about 19% (w/w), or about 20% (w/w). In certain embodiments, the amount of the microcrystalline cellulose in a pharmaceutical formulation described herein is about 15% (w/w). In certain embodiments, the amount of the microcrystalline cellulose in a pharmaceutical formulation described herein is about 16% (w/w). In certain embodiments, the amount of the microcrystalline cellulose in a pharmaceutical formulation described herein is about 17% (w/w). In certain embodiments, the amount of the microcrystalline cellulose in a pharmaceutical formulation described herein is about 18% (w/w). In certain embodiments, the amount of the microcrystalline cellulose in a pharmaceutical formulation described herein is about 19% (w/w). In certain embodiments, the amount of the microcrystalline cellulose in a pharmaceutical formulation described herein is about 20% (w/w).

In certain embodiments, the amount of the microcrystalline cellulose in a pharmaceutical formulation described herein is about 55 mg to about 65 mg. In certain embodiments, the amount of the microcrystalline cellulose in a pharmaceutical formulation described herein is about 55 mg, about 55.5 mg, about 56 mg, about 56.5 mg, about 57 mg, about 57.5 mg, about 58 mg, about 58.5 mg, about 59 mg, about 59.5 mg, about 60 mg, about 60.5 mg, about 61 mg, about 61.5 mg, about 62 mg, about 62.5 mg, about 63 mg, about 63.5 mg, about 64 mg, about 64.5 mg, or about 65 mg. In certain embodiments, the amount of the microcrystalline cellulose in a pharmaceutical formulation described herein is about 60 mg. In certain embodiments, the amount of the microcrystalline cellulose in a pharmaceutical formulation described herein is about 60.5 mg. In certain embodiments, the amount of the microcrystalline cellulose in a pharmaceutical formulation described herein is about 61 mg. In certain embodiments, the amount of the microcrystalline cellulose in a pharmaceutical formulation described herein is about 61.5 mg. In certain embodiments, the amount of the microcrystalline cellulose in a pharmaceutical formulation described herein is about 62 mg. In certain embodiments, the amount of the microcrystalline cellulose in a pharmaceutical formulation described herein is about 62.5 mg. In certain embodiments, the amount of the microcrystalline cellulose in a pharmaceutical formulation described herein is about 63 mg. In certain embodiments, the amount of the microcrystalline cellulose in a pharmaceutical formulation described herein is about 63 mg. In certain embodiments, the amount of the microcrystalline cellulose in a pharmaceutical formulation described herein is about 63.5 mg. In certain embodiments, the amount of the microcrystalline cellulose in a pharmaceutical formulation described herein is about 64 mg. In certain embodiments, the amount of the microcrystalline cellulose in a pharmaceutical formulation described herein is about 64.5 mg. In certain embodiments, the amount of the microcrystalline cellulose in a pharmaceutical formulation described herein is about 65 mg.

In certain embodiments, the amount of sodium starch glycolate in a pharmaceutical formulation described herein is about 6.5% (w/w) to about 7.5% (w/w). In certain embodiments, the amount of sodium starch glycolate in a pharmaceutical formulation described herein is about 6.5% (w/w), about 6.6% (w/w), about 6.7% (w/w), about 6.8% (w/w), about 6.9% (w/w), about 7% (w/w), about 7.1% (w/w), about 7.2% (w/w), about 7.3% (w/w), about 7.4% (w/w), or about 7.5% (w/w). In certain embodiments, the amount of sodium starch glycolate in a pharmaceutical formulation described herein is about 6.8% (w/w). In certain embodiments, the amount of sodium starch glycolate in a pharmaceutical formulation described herein is about 6.9% (w/w). In certain embodiments, the amount of sodium starch glycolate in a pharmaceutical formulation described herein is about 7% (w/w). In certain embodiments, the amount of sodium starch glycolate in a pharmaceutical formulation described herein is about 7.1% (w/w). In certain embodiments, the amount of sodium starch glycolate in a pharmaceutical formulation described herein is about 7.2% (w/w). In certain embodiments, the amount of sodium starch glycolate in a pharmaceutical formulation described herein is about 7.3% (w/w).

In certain embodiments, the amount of sodium starch glycolate in a pharmaceutical formulation described herein is about 20 mg to about 30 mg. In certain embodiments, the amount of sodium starch glycolate in a pharmaceutical formulation described herein is about 20 mg, about 21 mg, about 22 mg, about 23 mg, about 24 mg, about 25 mg, about 26 mg, about 27 mg, about 28 mg, about 29 mg, or about 30 mg. In certain embodiments, the amount of sodium starch glycolate in a pharmaceutical formulation described herein is about 22 mg. In certain embodiments, the amount of sodium starch glycolate in a pharmaceutical formulation described herein is about 23 mg. In certain embodiments, the amount of sodium starch glycolate in a pharmaceutical formulation described herein is about 24 mg. In certain embodiments, the amount of sodium starch glycolate in a pharmaceutical formulation described herein is about 25 mg. In certain embodiments, the amount of sodium starch glycolate in a pharmaceutical formulation described herein is about 26 mg. In certain embodiments, the amount of sodium starch glycolate in a pharmaceutical formulation described herein is about 27 mg. In certain embodiments, the amount of sodium starch glycolate in a pharmaceutical formulation described herein is about 28 mg.

In certain embodiments, the amount of the povidone in a pharmaceutical formulation described herein is 0% (w/w) to about 0.5% (w/w). In certain embodiments, the amount of the povidone in a pharmaceutical formulation described herein is about 0.1% (w/w), about 0.2% (w/w), about 0.3% (w/w), about 0.4% (w/w), or about 0.5% (w/w). In certain embodiments, the amount of the povidone in a pharmaceutical formulation described herein is about 0.1% (w/w). In certain embodiments, the amount of the povidone in a pharmaceutical formulation described herein is about 0.2% (w/w). In certain embodiments, the amount of the povidone in a pharmaceutical formulation described herein is about 0.3% (w/w). In certain embodiments, the amount of the povidone in a pharmaceutical formulation described herein is about 0.4% (w/w). In certain embodiments, the amount of the povidone in a pharmaceutical formulation described herein is about 0.5% (w/w).

In certain embodiments, the amount of the povidone in a pharmaceutical formulation described herein is 0 mg to about 1 mg. In certain embodiments, the amount of the povidone in a pharmaceutical formulation described herein is about 0.1 mg, about 0.2 mg, about 0.3 mg, about 0.4 mg, about 0.5 mg, about 0.6 mg, about 0.7 mg, about 0.8 mg, about 0.9 mg, or about 1 mg. In certain embodiments, the amount of the povidone in a pharmaceutical formulation described herein is about 0.6 mg. In certain embodiments, the amount of the povidone in a pharmaceutical formulation described herein is about 0.7 mg. In certain embodiments, the amount of the povidone in a pharmaceutical formulation described herein is about 0.8 mg. In certain embodiments, the amount of the povidone in a pharmaceutical formulation described herein is about 0.9 mg. In certain embodiments, the amount of the povidone in a pharmaceutical formulation described herein is about 1 mg.

In certain embodiments, the amount of sodium lauryl sulfate in a pharmaceutical formulation described herein is about 0% (w/w) to about 0.8% (w/w). In certain embodiments, the amount of sodium lauryl sulfate in a pharmaceutical formulation described herein is about 0.3% (w/w), about 0.4% (w/w), about 0.5% (w/w), about 0.6% (w/w), about 0.7% (w/w), or about 0.8% (w/w). In certain embodiments, the amount of sodium lauryl sulfate in a pharmaceutical formulation described herein is about 0.3% (w/w). In certain embodiments, the amount of sodium lauryl sulfate in a pharmaceutical formulation described herein is about 0.4% (w/w). In certain embodiments, the amount of sodium lauryl sulfate in a pharmaceutical formulation described herein is about 0.5% (w/w). In certain embodiments, the amount of sodium lauryl sulfate in a pharmaceutical formulation described herein is about 0.6% (w/w).

In certain embodiments, the amount of sodium lauryl sulfate in a pharmaceutical formulation described herein is 0 mg to about 2.5 mg. In certain embodiments, the amount of sodium lauryl sulfate in a pharmaceutical formulation described herein is about 1.5 mg, about 1.6 mg, about 1.7 mg, about 1.8 mg, about 1.9 mg, about 2 mg, about 2.1 mg, about 2.2 mg, about 2.3 mg, about 2.4 mg, or about 2.5 mg. In certain embodiments, the amount of sodium lauryl sulfate in a pharmaceutical formulation described herein is about 1.7 mg. In certain embodiments, the amount of sodium lauryl sulfate in a pharmaceutical formulation described herein is about 1.8 mg. In certain embodiments, the amount of sodium lauryl sulfate in a pharmaceutical formulation described herein is about 1.9 mg. In certain embodiments, the amount of sodium lauryl sulfate in a pharmaceutical formulation described herein is about 2 mg. In certain embodiments, the amount of sodium lauryl sulfate in a pharmaceutical formulation described herein is about 2.1 mg. In certain embodiments, the amount of sodium lauryl sulfate in a pharmaceutical formulation described herein is about 2.2 mg.

In various embodiments, provided herein is a pharmaceutical composition comprising
(i) about 45% (w/w) to about 55% (w/w) bempedoic acid;
(ii) about 1% (w/w) to about 2% (w/w) colloidal silicon dioxide;
(iii) about 3% (w/w) to about 5% (w/w) of a hydroxy propyl cellulose;
(iv) about 8% (w/w) to about 20% (w/w) lactose monohydrate;
(v) about 1% (w/w) to about 3% (w/w) magnesium stearate;
(vi) about 15% (w/w) to about 20% (w/w) a microcrystalline cellulose; and
(vi) about 6.5% (w/w) to about 7.5% (w/w) sodium starch glycolate.

In various embodiments, provided herein is a pharmaceutical composition comprising
(i) about 45% (w/w) to about 55% (w/w) bempedoic acid;
(ii) about 1% (w/w) to about 2% (w/w) colloidal silicon dioxide;
(iii) about 3% (w/w) to about 5% (w/w) of a hydroxy propyl cellulose;
(iv) about 8% (w/w) to about 20% (w/w) lactose monohydrate;
(v) about 1% (w/w) to about 3% (w/w) magnesium stearate;
(vi) about 15% (w/w) to about 20% (w/w) a microcrystalline cellulose;
(vi) about 6.5% (w/w) to about 7.5% (w/w) sodium starch glycolate;
(vii) 0% (w/w) to about 3% (w/w) ezetimibe;
(viii) 0% (w/w) to about 0.5% (w/w) of a povidone; and
(ix) 0% (w/w) to about 0.8% (w/w) sodium lauryl sulfate.

In various embodiments, provided herein is a pharmaceutical composition comprising
(i) 180 mg bempedoic acid;
(ii) about 3 mg to about 6 mg colloidal silicon dioxide;
(iii) about 10 mg to about 20 mg of a hydroxy propyl cellulose;
(iv) about 20 mg to about 80 mg lactose monohydrate;
(v) about 2 mg to about 10 mg magnesium stearate;
(vi) about 55 mg to about 65 mg a microcrystalline cellulose; and
(vi) about 20 mg to about 30 mg sodium starch glycolate.

In various embodiments, provided herein is a pharmaceutical composition comprising
(i) 180 mg bempedoic acid;
(ii) about 3 mg to about 6 mg colloidal silicon dioxide;
(iii) about 10 mg to about 20 mg of a hydroxy propyl cellulose;
(iv) about 20 mg to about 80 mg lactose monohydrate;
(v) about 2 mg to about 10 mg magnesium stearate;
(vi) about 55 mg to about 65 mg a microcrystalline cellulose;
(vi) about 20 mg to about 30 mg sodium starch glycolate;
(vii) 0 mg to about 10 mg ezetimibe;
(viii) 0 mg to about 1 mg of a povidone; and
(ix) 0 mg to about 2.5 mg sodium lauryl sulfate.

In certain embodiments, the pharmaceutical formulation further comprises a coating. In certain embodiments, the coating comprises Opadry White 85F18422. In certain embodiments, the coating comprises Opadry AMB II Blue.

In certain embodiments, the pharmaceutical compositions described herein may be administered in a unit dosage form and may be prepared by any method well known in the art of pharmacy.

In another aspect, provided herein are solid dosage forms comprising a pharmaceutical formulation described herein. In certain embodiments, the solid dosage forms described herein to be used for oral administration.

In various embodiments, the pharmaceutical formulation described herein is formulated for oral delivery. In certain embodiments, the pharmaceutical formulations described herein are formulated as an oral dosage form. Examples of oral dosage forms include, but are not limited to a drench, a tablet, a capsule, a softgel capsule, a cachet, a pill, an emulsion, a lozenge, a solution, a suspension, a bolus, a powder, an elixir or syrup, a pastille, a mouthwash, a granule, or a paste for application to the tongue. In some embodiments, the pharmaceutical composition is formulated as a tablet.

In certain embodiments, a pharmaceutical composition described herein can be used for the treatment of a disease, disorder, or condition described herein.

### Methods of Use and Treatments

Provided herein are methods of reducing the risk of myocardial infarction or coronary revascularization in an adult with established cardiovascular disease or two or more risk factors for cardiovascular disease. Also provided herein are methods of reducing low-density lipoprotein cholesterol (LDL-C) in an adult with primary hyperlipidemia.

### (1) Myocardial Infarction

In one aspect, provided herein are methods of reducing the risk of myocardial infarction in an adult with established cardiovascular disease or two or more risk factors for cardiovascular disease, the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of reducing the risk of myocardial infarction in an adult with established cardiovascular disease or two or more risk factors for cardiovascular disease, the method comprising administering to the adult a pharmaceutical formulation described herein.

In various embodiments, provided herein is a method of reducing the risk of myocardial infarction in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof.

In certain embodiments, the myocardial infarction is nonfatal myocardial infarction.

In certain embodiments, the adult has established cardiovascular disease.

In certain embodiments, the adult has two or more risk factors for cardiovascular disease. In certain embodiments, the two or more risk factors for cardiovascular disease are selected from the group consisting of history of tobacco use such as cigarette smoking, diabetes mellitus (DM) hypertension, high body mass index (BMI), obesity, and dyslipidemia. Other risk factors can include age, high blood pressure, unhealthy diet, physical inactivity, a coronary calcium score greater than 400 (Agatston score), and the presence of atherosclerosis (as determined, for example, by an angiogram, an ultrasound of the carotid arteries, and/or computed tomography of the coronary arteries).

In certain embodiments, the adult has primary hyperlipidemia. In certain embodiments, the primary hyperlipidemia is heterozygous familial hypercholesterolemia.

In certain embodiments, the pharmaceutical formulation comprises colloidal silicon dioxide; hydroxyl propyl cellulose; lactose monohydrate; magnesium stearate; microcrystalline cellulose; and sodium starch glycolate. In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the pharmaceutical formulation further comprises 10 mg ezetimibe.

In certain embodiments, the method further comprises administering to the adult another low-density lipoprotein cholesterol (LDL-C) lowering therapy (which also can be referred to herein as simply a “LDL-C lowering therapy”).

In certain embodiments, the LDL-C-lowering therapy comprises niacin, a bile acid resin, a fibrate, or a proprotein convertase subtilisin kexin type 9 (PCSK9) inhibitor.

In certain embodiments, the LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering an LDL-C lowering therapy comprises administering to the adult about 10 mg to about 80 mg atorvastatin. In certain embodiments, administering an LDL-C lowering therapy comprises administering to the adult 10 mg to 80 mg atorvastatin.

In certain embodiments, administering an LDL-C lowering therapy comprises administering to the adult about 10 mg, about 20 mg, about 40 mg, or about 80 mg atorvastatin. In certain embodiments, administering an LDL-C lowering therapy comprises administering to the adult 10 mg, 20 mg, 40 mg, or 80 mg atorvastatin. In certain embodiments, administering an LDL-C lowering therapy comprises administering to the adult about 10 mg atorvastatin. In certain embodiments, administering an LDL-C lowering therapy comprises administering to the adult about 20 mg atorvastatin. In certain embodiments, administering an LDL-C lowering therapy comprises administering to the adult about 40 mg atorvastatin. In certain embodiments, administering an LDL-C lowering therapy comprises administering to the adult about 80 mg atorvastatin. In certain embodiments, administering an LDL-C lowering therapy comprises administering to the adult 10 mg atorvastatin. In certain embodiments, administering an LDL-C lowering therapy comprises administering to the adult 20 mg atorvastatin. In certain embodiments, administering an LDL-C lowering therapy comprises administering to the adult 40 mg atorvastatin. In certain embodiments, administering an LDL-C lowering therapy comprises administering to the adult 80 mg atorvastatin.

In certain embodiments, administering an LDL-C lowering therapy comprises administering to the adult about 20 mg to about 80 mg fluvastatin. In certain embodiments, administering an LDL-C lowering therapy comprises administering to the adult 20 mg to 80 mg fluvastatin.

In certain embodiments, administering an LDL-C lowering therapy comprises administering to the adult about 20 mg, about 40 mg, or about 80 mg fluvastatin. In certain embodiments, administering an LDL-C lowering therapy comprises administering to the adult 20 mg, 40 mg, or 80 mg fluvastatin. In certain embodiments, administering an LDL-C lowering therapy comprises administering to the adult about 20 mg fluvastatin. In certain embodiments, administering an LDL-C lowering therapy comprises administering to the adult about 40 mg fluvastatin. In certain embodiments, administering an LDL-C lowering therapy comprises administering to the adult about 80 mg fluvastatin. In certain embodiments, administering an LDL-C lowering therapy comprises administering to the adult 20 mg fluvastatin. In certain embodiments, administering an LDL-C lowering therapy comprises administering to the adult 40 mg fluvastatin. In certain embodiments, administering an LDL-C lowering therapy comprises administering to the adult 80 mg fluvastatin.

In certain embodiments, administering an LDL-C lowering therapy comprises administering to the adult about 20 mg to about 40 mg lovastatin. In certain embodiments, administering an LDL-C lowering therapy comprises administering to the adult 20 mg to 40 mg lovastatin.

In certain embodiments, administering an LDL-C lowering therapy comprises administering to the adult about 20 mg or about 40 mg lovastatin. In certain embodiments, administering an LDL-C lowering therapy comprises administering to the adult 20 mg or 40 mg lovastatin. In certain embodiments, administering an LDL-C lowering therapy comprises administering to the adult about 20 mg lovastatin. In certain embodiments, administering an LDL-C lowering therapy comprises administering to the adult about 40 mg lovastatin. In certain embodiments, administering an LDL-C lowering therapy comprises administering to the adult 20 mg lovastatin. In certain embodiments, administering an LDL-C lowering therapy comprises administering to the adult 40 mg lovastatin.

In certain embodiments, administering an LDL-C lowering therapy comprises administering to the adult about 1 mg to about 4 mg pitavastatin. In certain embodiments, administering an LDL-C lowering therapy comprises administering to the adult 1 mg to 4 mg pitavastatin.

In certain embodiments, administering an LDL-C lowering therapy comprises administering to the adult about 1 mg, about 2 mg, or about 4 mg pitavastatin. In certain embodiments, administering an LDL-C lowering therapy comprises administering to the adult 1 mg, 2 mg, or 4 mg pitavastatin. In certain embodiments, administering an LDL-C lowering therapy comprises administering to the adult about 1 mg pitavastatin. In certain embodiments, administering an LDL-C lowering therapy comprises administering to the adult about 2 mg pitavastatin. In certain embodiments, administering an LDL-C lowering therapy comprises administering to the adult about 4 mg pitavastatin. In certain embodiments, administering an LDL-C lowering therapy comprises administering to the adult 1 mg pitavastatin. In certain embodiments, administering an LDL-C lowering therapy comprises administering to the adult 2 mg pitavastatin. In certain embodiments, administering an LDL-C lowering therapy comprises administering to the adult 4 mg pitavastatin.

In certain embodiments, administering an LDL-C lowering therapy comprises administering to the adult about 10 mg to about 40 mg pravastatin. In certain embodiments, administering an LDL-C lowering therapy comprises administering to the adult 10 mg to 40 mg pravastatin.

In certain embodiments, administering an LDL-C lowering therapy comprises administering to the adult about 10 mg, about 20 mg, or about 40 mg pravastatin. In certain embodiments, administering an LDL-C lowering therapy comprises administering to the adult 10 mg, 20 mg, or 40 mg pravastatin. In certain embodiments, administering an LDL-C lowering therapy comprises administering to the adult about 10 mg pravastatin. In certain embodiments, administering an LDL-C lowering therapy comprises administering to the adult about 20 mg pravastatin. In certain embodiments, administering an LDL-C lowering therapy comprises administering to the adult about 40 mg pravastatin. In certain embodiments, administering an LDL-C lowering therapy comprises administering to the adult 10 mg pravastatin. In certain embodiments, administering an LDL-C lowering therapy comprises administering to the adult 20 mg pravastatin. In certain embodiments, administering an LDL-C lowering therapy comprises administering to the adult 40 mg pravastatin.

In certain embodiments, administering an LDL-C lowering therapy comprises administering to the adult about 5 mg to about 40 mg rosuvastatin. In certain embodiments, administering an LDL-C lowering therapy comprises administering to the adult 5 mg to 40 mg rosuvastatin.

In certain embodiments, administering an LDL-C lowering therapy comprises administering to the adult about 5 mg, about 10 mg, about 20 mg, or about 40 mg rosuvastatin. In certain embodiments, administering an LDL-C lowering therapy comprises administering to the adult 5 mg, 10 mg, 20 mg, or 40 mg rosuvastatin. In certain embodiments, administering an LDL-C lowering therapy comprises administering to the adult about 5 mg rosuvastatin. In certain embodiments, administering an LDL-C lowering therapy comprises administering to the adult about 10 mg rosuvastatin. In certain embodiments, administering an LDL-C lowering therapy comprises administering to the adult about 20 mg rosuvastatin. In certain embodiments, administering an LDL-C lowering therapy comprises administering to the adult about 40 mg rosuvastatin. In certain embodiments, administering an LDL-C lowering therapy comprises administering to the adult 5 mg rosuvastatin. In certain embodiments, administering an LDL-C lowering therapy comprises administering to the adult 10 mg rosuvastatin. In certain embodiments, administering an LDL-C lowering therapy comprises administering to the adult 20 mg rosuvastatin. In certain embodiments, administering an LDL-C lowering therapy comprises administering to the adult 40 mg rosuvastatin.

In certain embodiments, administering an LDL-C lowering therapy comprises administering to the adult about 10 mg to about 20 mg simvastatin. In certain embodiments, administering an LDL-C lowering therapy comprises administering to the adult 10 mg to 20 mg simvastatin.

In certain embodiments, administering an LDL-C lowering therapy comprises administering to the adult about 10 mg or about 20 mg simvastatin. In certain embodiments, administering an LDL-C lowering therapy comprises administering to the adult 10 mg or 20 mg. In certain embodiments, administering an LDL-C lowering therapy comprises administering to the adult about 10 mg simvastatin. In certain embodiments, administering an LDL-C lowering therapy comprises administering to the adult about 20 mg simvastatin. In certain embodiments, administering an LDL-C lowering therapy comprises administering to the adult 10 mg simvastatin. In certain embodiments, administering an LDL-C lowering therapy comprises administering to the adult 20 mg simvastatin.

In certain embodiments, the LDL-C-lowering therapy comprises ezetimibe.

In certain embodiments, administering an LDL-C lowering therapy comprises administering to the adult about 10 mg ezetimibe. In certain embodiments, administering an LDL-C lowering therapy comprises administering to the adult 10 mg ezetimibe. In certain embodiments, administering an LDL-C lowering therapy comprises administering to the adult orally 10 mg ezetimibe. In certain embodiments, administering an LDL-C lowering therapy comprises administering to the adult orally 10 mg ezetimibe once daily.

In certain embodiments, the LDL-C lowering therapy is administered to the adult once, twice, three, four, five, six, or seven times a week. In certain embodiments, the LDL-C lowering therapy is administered to the adult once, twice, three, four, or five times daily. In certain embodiments, the LDL-C lowering therapy is administered to the adult once daily. In certain embodiments, the LDL-C lowering therapy is administered to the adult twice daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (2) Coronary Revascularization

In one aspect, provided herein are methods of reducing the risk of coronary revascularization in an adult with established cardiovascular disease or two or more risk factors for cardiovascular disease, the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of reducing the risk of coronary revascularization in an adult with established cardiovascular disease or two or more risk factors for cardiovascular disease, the method comprising administering to the adult a pharmaceutical formulation described herein.

In various embodiments, provided herein is a method of reducing the risk of coronary revascularization in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof.

In certain embodiments, the adult has established cardiovascular disease.

In certain embodiments, the adult has two or more risk factors for cardiovascular disease. In certain embodiments, the two or more risk factors for cardiovascular disease are selected from the group consisting of history of tobacco use such as cigarette smoking, diabetes mellitus (DM) hypertension, high body mass index (BMI), obesity, and dyslipidemia. Other risk factors can include age, high blood pressure, unhealthy diet, physical inactivity, a coronary calcium score greater than 400 (Agatston score), and the presence of atherosclerosis (as determined, for example, by an angiogram, an ultrasound of the carotid arteries, and/or computed tomography of the coronary arteries).

In certain embodiments, the adult has primary hyperlipidemia. In certain embodiments, the primary hyperlipidemia is heterozygous familial hypercholesterolemia.

In certain embodiments, the pharmaceutical formulation comprises colloidal silicon dioxide; hydroxyl propyl cellulose; lactose monohydrate; magnesium stearate; microcrystalline cellulose; and sodium starch glycolate. In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the pharmaceutical formulation further comprises 10 mg ezetimibe.

In certain embodiments, the method further comprises administering to the adult a low-density lipoprotein cholesterol (LDL-C) lowering therapy.

In certain embodiments, the LDL-C-lowering therapy comprises niacin, a bile acid resin, a fibrate, or a proprotein convertase subtilisin kexin type 9 (PCSK9) inhibitor.

In certain embodiments, the LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering an LDL-C lowering therapy comprises administering to the adult about 10 mg to about 80 mg atorvastatin. In certain embodiments, administering an LDL-C lowering therapy comprises administering to the adult 10 mg to 80 mg atorvastatin.

In certain embodiments, administering an LDL-C lowering therapy comprises administering to the adult about 10 mg, about 20 mg, about 40 mg, or about 80 mg atorvastatin. In certain embodiments, administering an LDL-C lowering therapy comprises administering to the adult 10 mg, 20 mg, 40 mg, or 80 mg atorvastatin. In certain embodiments, administering an LDL-C lowering therapy comprises administering to the adult about 10 mg atorvastatin. In certain embodiments, administering an LDL-C lowering therapy comprises administering to the adult about 20 mg atorvastatin. In certain embodiments, administering an LDL-C lowering therapy comprises administering to the adult about 40 mg atorvastatin. In certain embodiments, administering an LDL-C lowering therapy comprises administering to the adult about 80 mg atorvastatin. In certain embodiments, administering an LDL-C lowering therapy comprises administering to the adult 10 mg atorvastatin. In certain embodiments, administering an LDL-C lowering therapy comprises administering to the adult 20 mg atorvastatin. In certain embodiments, administering an LDL-C lowering therapy comprises administering to the adult 40 mg atorvastatin. In certain embodiments, administering an LDL-C lowering therapy comprises administering to the adult 80 mg atorvastatin.

In certain embodiments, administering an LDL-C lowering therapy comprises administering to the adult about 20 mg to about 80 mg fluvastatin. In certain embodiments, administering an LDL-C lowering therapy comprises administering to the adult 20 mg to 80 mg fluvastatin.

In certain embodiments, administering an LDL-C lowering therapy comprises administering to the adult about 20 mg, about 40 mg, or about 80 mg fluvastatin. In certain embodiments, administering an LDL-C lowering therapy comprises administering to the adult 20 mg, 40 mg, or 80 mg fluvastatin. In certain embodiments, administering an LDL-C lowering therapy comprises administering to the adult about 20 mg fluvastatin. In certain embodiments, administering an LDL-C lowering therapy comprises administering to the adult about 40 mg fluvastatin. In certain embodiments, administering an LDL-C lowering therapy comprises administering to the adult about 80 mg fluvastatin. In certain embodiments, administering an LDL-C lowering therapy comprises administering to the adult 20 mg fluvastatin. In certain embodiments, administering an LDL-C lowering therapy comprises administering to the adult 40 mg fluvastatin. In certain embodiments, administering an LDL-C lowering therapy comprises administering to the adult 80 mg fluvastatin.

In certain embodiments, administering an LDL-C lowering therapy comprises administering to the adult about 20 mg to about 40 mg lovastatin. In certain embodiments, administering an LDL-C lowering therapy comprises administering to the adult 20 mg to 40 mg lovastatin.

In certain embodiments, administering an LDL-C lowering therapy comprises administering to the adult about 20 mg or about 40 mg lovastatin. In certain embodiments, administering an LDL-C lowering therapy comprises administering to the adult 20 mg or 40 mg lovastatin. In certain embodiments, administering an LDL-C lowering therapy comprises administering to the adult about 20 mg lovastatin. In certain embodiments, administering an LDL-C lowering therapy comprises administering to the adult about 40 mg lovastatin. In certain embodiments, administering an LDL-C lowering therapy comprises administering to the adult 20 mg lovastatin. In certain embodiments, administering an LDL-C lowering therapy comprises administering to the adult 40 mg lovastatin.

In certain embodiments, administering an LDL-C lowering therapy comprises administering to the adult about 1 mg to about 4 mg pitavastatin. In certain embodiments, administering an LDL-C lowering therapy comprises administering to the adult 1 mg to 4 mg pitavastatin.

In certain embodiments, administering an LDL-C lowering therapy comprises administering to the adult about 1 mg, about 2 mg, or about 4 mg pitavastatin. In certain embodiments, administering an LDL-C lowering therapy comprises administering to the adult 1 mg, 2 mg, or 4 mg pitavastatin. In certain embodiments, administering an LDL-C lowering therapy comprises administering to the adult about 1 mg pitavastatin. In certain embodiments, administering an LDL-C lowering therapy comprises administering to the adult about 2 mg pitavastatin. In certain embodiments, administering an LDL-C lowering therapy comprises administering to the adult about 4 mg pitavastatin. In certain embodiments, administering an LDL-C lowering therapy comprises administering to the adult 1 mg pitavastatin. In certain embodiments, administering an LDL-C lowering therapy comprises administering to the adult 2 mg pitavastatin. In certain embodiments, administering an LDL-C lowering therapy comprises administering to the adult 4 mg pitavastatin.

In certain embodiments, administering an LDL-C lowering therapy comprises administering to the adult about 10 mg to about 40 mg pravastatin. In certain embodiments, administering an LDL-C lowering therapy comprises administering to the adult 10 mg to 40 mg pravastatin.

In certain embodiments, administering an LDL-C lowering therapy comprises administering to the adult about 10 mg, about 20 mg, or about 40 mg pravastatin. In certain embodiments, administering an LDL-C lowering therapy comprises administering to the adult 10 mg, 20 mg, or 40 mg pravastatin. In certain embodiments, administering an LDL-C lowering therapy comprises administering to the adult about 10 mg pravastatin. In certain embodiments, administering an LDL-C lowering therapy comprises administering to the adult about 20 mg pravastatin. In certain embodiments, administering an LDL-C lowering therapy comprises administering to the adult about 40 mg pravastatin. In certain embodiments, administering an LDL-C lowering therapy comprises administering to the adult 10 mg pravastatin. In certain embodiments, administering an LDL-C lowering therapy comprises administering to the adult 20 mg pravastatin. In certain embodiments, administering an LDL-C lowering therapy comprises administering to the adult 40 mg pravastatin.

In certain embodiments, administering an LDL-C lowering therapy comprises administering to the adult about 5 mg to about 40 mg rosuvastatin. In certain embodiments, administering an LDL-C lowering therapy comprises administering to the adult 5 mg to 40 mg rosuvastatin.

In certain embodiments, administering an LDL-C lowering therapy comprises administering to the adult about 5 mg, about 10 mg, about 20 mg, or about 40 mg rosuvastatin. In certain embodiments, administering an LDL-C lowering therapy comprises administering to the adult 5 mg, 10 mg, 20 mg, or 40 mg rosuvastatin. In certain embodiments, administering an LDL-C lowering therapy comprises administering to the adult about 5 mg rosuvastatin. In certain embodiments, administering an LDL-C lowering therapy comprises administering to the adult about 10 mg rosuvastatin. In certain embodiments, administering an LDL-C lowering therapy comprises administering to the adult about 20 mg rosuvastatin. In certain embodiments, administering an LDL-C lowering therapy comprises administering to the adult about 40 mg rosuvastatin. In certain embodiments, administering an LDL-C lowering therapy comprises administering to the adult 5 mg rosuvastatin. In certain embodiments, administering an LDL-C lowering therapy comprises administering to the adult 10 mg rosuvastatin. In certain embodiments, administering an LDL-C lowering therapy comprises administering to the adult 20 mg rosuvastatin. In certain embodiments, administering an LDL-C lowering therapy comprises administering to the adult 40 mg rosuvastatin.

In certain embodiments, administering an LDL-C lowering therapy comprises administering to the adult about 10 mg to about 20 mg simvastatin. In certain embodiments, administering an LDL-C lowering therapy comprises administering to the adult 10 mg to 20 mg simvastatin.

In certain embodiments, administering an LDL-C lowering therapy comprises administering to the adult about 10 mg or about 20 mg simvastatin. In certain embodiments, administering an LDL-C lowering therapy comprises administering to the adult 10 mg or 20 mg. In certain embodiments, administering an LDL-C lowering therapy comprises administering to the adult about 10 mg simvastatin. In certain embodiments, administering an LDL-C lowering therapy comprises administering to the adult about 20 mg simvastatin. In certain embodiments, administering an LDL-C lowering therapy comprises administering to the adult 10 mg simvastatin. In certain embodiments, administering an LDL-C lowering therapy comprises administering to the adult 20 mg simvastatin.

In certain embodiments, the LDL-C-lowering therapy comprises ezetimibe.

In certain embodiments, administering an LDL-C lowering therapy comprises administering to the adult about 10 mg ezetimibe. In certain embodiments, administering an LDL-C lowering therapy comprises administering to the adult 10 mg ezetimibe.

In certain embodiments, the LDL-C lowering therapy is administered to the adult once, twice, three, four, five, six, or seven times a week. In certain embodiments, the LDL-C lowering therapy is administered to the adult once, twice, three, four, or five times daily. In certain embodiments, the LDL-C lowering therapy is administered to the adult once daily. In certain embodiments, the LDL-C lowering therapy is administered to the adult twice daily.

In certain embodiments, the LDL-C-lowering therapy comprises ezetimibe. In certain embodiments, administering an LDL-C lowering therapy comprises administering to the adult 10 mg ezetimibe. In certain embodiments, administering an LDL-C lowering therapy comprises administering to the adult orally 10 mg ezetimibe. In certain embodiments, administering an LDL-C lowering therapy comprises administering to the adult orally 10 mg ezetimibe once daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (3) Primary Hyperlipidemia

In one aspect, provided herein are methods of reducing low-density lipoprotein cholesterol (LDL-C) in an adult with primary hyperlipidemia, the methods generally comprise administering to the adult an effective amount of bempedoic acid (e.g., a pharmaceutical material comprising bempedoic acid described herein and/or a crystalline form of bempedoic acid described herein).

In certain embodiments, the effective amount of bempedoic acid is administered to the adult daily. In certain embodiments, the effective amount of bempedoic acid is administered to the adult once, twice, three, four, or five times daily. In certain embodiments, the effective amount of bempedoic acid is administered to the adult once daily.

In certain embodiments, the effective amount of bempedoic acid is administered to the adult orally.

In certain embodiments, administering an effective amount of bempedoic acid comprises administering to the adult 180 mg bempedoic acid. In certain embodiments, administering an effective amount of bempedoic acid comprises administering to the adult orally 180 mg bempedoic acid daily. In certain embodiments, administering an effective amount of bempedoic acid comprises administering to the adult orally 180 mg bempedoic acid once daily.

In various embodiments, provided herein is a method of reducing low-density lipoprotein cholesterol (LDL-C) in an adult with primary hyperlipidemia, the method comprising administering to the adult a pharmaceutical formulation described herein.

In various embodiments, provided herein is a method of reducing low-density lipoprotein cholesterol (LDL-C) in an adult with primary hyperlipidemia, the method comprising administering to the adult a fixed-dose combination described herein.

In various embodiments, provided herein is a method of reducing LDL-C in an adult with primary hyperlipidemia, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof, alone or in combination with another LDL-C-lowering therapy.

In certain embodiments, the pharmaceutical formulation comprises colloidal silicon dioxide; hydroxyl propyl cellulose; lactose monohydrate; magnesium stearate; microcrystalline cellulose; and sodium starch glycolate.

In certain embodiments, the other LDL-C-lowering therapy comprises ezetimibe. In certain embodiments, administering the other LDL-C-lowering therapy comprises administering to the adult 10 mg ezetimibe. In certain embodiments, administering the other LDL-C-lowering therapy comprises administering to the adult orally 10 mg ezetimibe. In certain embodiments, administering the other LDL-C-lowering therapy comprises administering to the adult orally 10 mg ezetimibe once daily.

In certain embodiments, the pharmaceutical formulation further comprises 10 mg ezetimibe.

In various embodiments, provided herein is a method of reducing LDL-C in an adult with primary hyperlipidemia, the method comprising orally administering daily to the adult a pharmaceutical formulation comprising 180 mg bempedoic acid; 10 mg ezetimibe; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof, alone or in combination with another LDL-C-lowering therapy.

In certain embodiments, the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

In certain embodiments, the primary hyperlipidemia is heterozygous familial hypercholesterolemia.

In certain embodiments, the other LDL-C-lowering therapy comprises niacin, a bile acid resin, a fibrate, or a proprotein convertase subtilisin kexin type 9 (PCSK9) inhibitor.

In certain embodiments, the other LDL-C-lowering therapy comprises a statin. In certain embodiments, the statin is selected from the group consisting of atorvastatin, fluvastatin, lovastatin, pitavastatin, pravastatin, rosuvastatin, simvastatin, and combinations thereof.

In certain embodiments, administering the other LDL-C lowering therapy comprises administering to the adult about 10 mg to about 80 mg atorvastatin. In certain embodiments, administering the other LDL-C lowering therapy comprises administering to the adult 10 mg to 80 mg atorvastatin.

In certain embodiments, administering the other LDL-C lowering therapy comprises administering to the adult about 10 mg, about 20 mg, about 40 mg, or about 80 mg atorvastatin. In certain embodiments, administering the other LDL-C lowering therapy comprises administering to the adult 10 mg, 20 mg, 40 mg, or 80 mg atorvastatin. In certain embodiments, administering the other LDL-C lowering therapy comprises administering to the adult about 10 mg atorvastatin. In certain embodiments, administering the other LDL-C lowering therapy comprises administering to the adult about 20 mg atorvastatin. In certain embodiments, administering the other LDL-C lowering therapy comprises administering to the adult about 40 mg atorvastatin. In certain embodiments, administering the other LDL-C lowering therapy comprises administering to the adult about 80 mg atorvastatin. In certain embodiments, administering the other LDL-C lowering therapy comprises administering to the adult 10 mg atorvastatin. In certain embodiments, administering the other LDL-C lowering therapy comprises administering to the adult 20 mg atorvastatin. In certain embodiments, administering the other LDL-C lowering therapy comprises administering to the adult 40 mg atorvastatin. In certain embodiments, administering the other LDL-C lowering therapy comprises administering to the adult 80 mg atorvastatin.

In certain embodiments, administering the other LDL-C lowering therapy comprises administering to the adult about 20 mg to about 80 mg fluvastatin. In certain embodiments, administering the other LDL-C lowering therapy comprises administering to the adult 20 mg to 80 mg fluvastatin.

In certain embodiments, administering the other LDL-C lowering therapy comprises administering to the adult about 20 mg, about 40 mg, or about 80 mg fluvastatin. In certain embodiments, administering the other LDL-C lowering therapy comprises administering to the adult 20 mg, 40 mg, or 80 mg fluvastatin. In certain embodiments, administering the other LDL-C lowering therapy comprises administering to the adult about 20 mg fluvastatin. In certain embodiments, administering the other LDL-C lowering therapy comprises administering to the adult about 40 mg fluvastatin. In certain embodiments, administering the other LDL-C lowering therapy comprises administering to the adult about 80 mg fluvastatin. In certain embodiments, administering the other LDL-C lowering therapy comprises administering to the adult 20 mg fluvastatin. In certain embodiments, administering the other LDL-C lowering therapy comprises administering to the adult 40 mg fluvastatin. In certain embodiments, administering the other LDL-C lowering therapy comprises administering to the adult 80 mg fluvastatin.

In certain embodiments, administering the other LDL-C lowering therapy comprises administering to the adult about 20 mg to about 40 mg lovastatin. In certain embodiments, administering the other LDL-C lowering therapy comprises administering to the adult 20 mg to 40 mg lovastatin.

In certain embodiments, administering the other LDL-C lowering therapy comprises administering to the adult about 20 mg or about 40 mg lovastatin. In certain embodiments, administering the other LDL-C lowering therapy comprises administering to the adult 20 mg or 40 mg lovastatin. In certain embodiments, administering the other LDL-C lowering therapy comprises administering to the adult about 20 mg lovastatin. In certain embodiments, administering the other LDL-C lowering therapy comprises administering to the adult about 40 mg lovastatin. In certain embodiments, administering the other LDL-C lowering therapy comprises administering to the adult 20 mg lovastatin. In certain embodiments, administering the other LDL-C lowering therapy comprises administering to the adult 40 mg lovastatin.

In certain embodiments, administering the other LDL-C lowering therapy comprises administering to the adult about 1 mg to about 4 mg pitavastatin. In certain embodiments, administering the other LDL-C lowering therapy comprises administering to the adult 1 mg to 4 mg pitavastatin.

In certain embodiments, administering the other LDL-C lowering therapy comprises administering to the adult about 1 mg, about 2 mg, or about 4 mg pitavastatin. In certain embodiments, administering the other LDL-C lowering therapy comprises administering to the adult 1 mg, 2 mg, or 4 mg pitavastatin. In certain embodiments, administering the other LDL-C lowering therapy comprises administering to the adult about 1 mg pitavastatin. In certain embodiments, administering the other LDL-C lowering therapy comprises administering to the adult about 2 mg pitavastatin. In certain embodiments, administering the other LDL-C lowering therapy comprises administering to the adult about 4 mg pitavastatin. In certain embodiments, administering the other LDL-C lowering therapy comprises administering to the adult 1 mg pitavastatin. In certain embodiments, administering the other LDL-C lowering therapy comprises administering to the adult 2 mg pitavastatin. In certain embodiments, administering the other LDL-C lowering therapy comprises administering to the adult 4 mg pitavastatin.

In certain embodiments, administering the other LDL-C lowering therapy comprises administering to the adult about 10 mg to about 40 mg pravastatin. In certain embodiments, administering the other LDL-C lowering therapy comprises administering to the adult 10 mg to 40 mg pravastatin.

In certain embodiments, administering the other LDL-C lowering therapy comprises administering to the adult about 10 mg, about 20 mg, or about 40 mg pravastatin. In certain embodiments, administering the other LDL-C lowering therapy comprises administering to the adult 10 mg, 20 mg, or 40 mg pravastatin. In certain embodiments, administering the other LDL-C lowering therapy comprises administering to the adult about 10 mg pravastatin. In certain embodiments, administering the other LDL-C lowering therapy comprises administering to the adult about 20 mg pravastatin. In certain embodiments, administering the other LDL-C lowering therapy comprises administering to the adult about 40 mg pravastatin. In certain embodiments, administering the other LDL-C lowering therapy comprises administering to the adult 10 mg pravastatin. In certain embodiments, administering the other LDL-C lowering therapy comprises administering to the adult 20 mg pravastatin. In certain embodiments, administering the other LDL-C lowering therapy comprises administering to the adult 40 mg pravastatin.

In certain embodiments, administering the other LDL-C lowering therapy comprises administering to the adult about 5 mg to about 40 mg rosuvastatin. In certain embodiments, administering the other LDL-C lowering therapy comprises administering to the adult 5 mg to 40 mg rosuvastatin.

In certain embodiments, administering the other LDL-C lowering therapy comprises administering to the adult about 5 mg, about 10 mg, about 20 mg, or about 40 mg rosuvastatin. In certain embodiments, administering the other LDL-C lowering therapy comprises administering to the adult 5 mg, 10 mg, 20 mg, or 40 mg rosuvastatin. In certain embodiments, administering the other LDL-C lowering therapy comprises administering to the adult about 5 mg rosuvastatin. In certain embodiments, administering the other LDL-C lowering therapy comprises administering to the adult about 10 mg rosuvastatin. In certain embodiments, administering the other LDL-C lowering therapy comprises administering to the adult about 20 mg rosuvastatin. In certain embodiments, administering the other LDL-C lowering therapy comprises administering to the adult about 40 mg rosuvastatin. In certain embodiments, administering the other LDL-C lowering therapy comprises administering to the adult 5 mg rosuvastatin. In certain embodiments, administering the other LDL-C lowering therapy comprises administering to the adult 10 mg rosuvastatin. In certain embodiments, administering the other LDL-C lowering therapy comprises administering to the adult 20 mg rosuvastatin. In certain embodiments, administering the other LDL-C lowering therapy comprises administering to the adult 40 mg rosuvastatin.

In certain embodiments, administering the other LDL-C lowering therapy comprises administering to the adult about 10 mg to about 20 mg simvastatin. In certain embodiments, administering the other LDL-C lowering therapy comprises administering to the adult 10 mg to 20 mg simvastatin.

In certain embodiments, administering the other LDL-C lowering therapy comprises administering to the adult about 10 mg or about 20 mg simvastatin. In certain embodiments, administering the other LDL-C lowering therapy comprises administering to the adult 10 mg or 20 mg. In certain embodiments, administering the other LDL-C lowering therapy comprises administering to the adult about 10 mg simvastatin. In certain embodiments, administering the other LDL-C lowering therapy comprises administering to the adult about 20 mg simvastatin. In certain embodiments, administering the other LDL-C lowering therapy comprises administering to the adult 10 mg simvastatin. In certain embodiments, administering the other LDL-C lowering therapy comprises administering to the adult 20 mg simvastatin.

In certain embodiments, the other LDL-C-lowering therapy comprises ezetimibe.

In certain embodiments, the LDL-C lowering therapy is administered to the adult once, twice, three, four, five, six, or seven times a week. In certain embodiments, the other LDL-C lowering therapy is administered to the subject once, twice, three, four, or five times daily. In certain embodiments, the other LDL-C lowering therapy is administered to the subject once daily. In certain embodiments, the other LDL-C lowering therapy is administered to the subject twice daily.

In certain embodiments, the adult is statin intolerant.

In certain embodiments, the pharmaceutical formulation is administered to the adult once daily.

In certain embodiments, the effective amount of bempedoic acid is administered to the adult once daily for 1, 2, 3, 4, 5, 6, 7, 8, 9, 10, 11, or 12 months. In certain embodiments, the effective amount of bempedoic acid is administered to the adult for the duration of the adult's lifespan.

In certain embodiments, the pharmaceutical formulation is administered to the adult once daily for 1, 2, 3, 4, 5, 6, 7, 8, 9, 10, 11, or 12 months. In certain embodiments, the pharmaceutical formulation is administered to the adult for the duration of the adult's lifespan.

### (4) Outcomes

In certain embodiments, the methods described herein decrease low-density lipoprotein cholesterol (LDL-C) in the adult about 21%, as compared to baseline (e.g., prior to initial administration of the effective amount of bempedoic acid).

In certain embodiments, the methods described herein decrease high-sensitivity C-reactive protein (hsCRP) about 21.6%, as compared to baseline (e.g., prior to the initial administration of the effective amount of bempedoic acid).

In certain embodiments, the methods described herein reduce the risk of myocardial infarction in the adult about 23%, as compared to an adult not receiving the effective amount of bempedoic acid therapy (e.g., 180 mg bempedoic acid).

In certain embodiments, the methods described herein reduce the risk of coronary revascularization in the adult about 19%, as compared to an adult not receiving the effective amount of bempedoic acid therapy (e.g., 180 mg bempedoic acid).

### EXAMPLES

Below are examples of specific embodiments for carrying out the present disclosure. The examples are offered for illustrative purposes only and are not intended to limit the scope of the present disclosure in any way.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

### Example 1—A Clinical Study of Bempedoic Acid and Cardiovascular Outcomes in Statin Intolerant Patients

### 1) Methods

### Trial Organization and Oversight

The CLEAR OUTCOMES trial was a randomized, placebo-controlled, double-blind trial that enrolled patients at 1250 sites in 32 countries. An independent Data Monitoring Committee reviewed safety and efficacy data during the trial.

### Trial Population

Patients 18 to 85 years of age were eligible for enrollment if they met either of two criteria for increased cardiovascular risk, a prior cardiovascular event (secondary prevention) or clinical features that placed them at high risk for a cardiovascular event (primary prevention). (Nicholls S, Lincoff A M, Bays H E, et al. Rationale and design of the CLEAR-outcomes trial: Evaluating the effect of bempedoic acid on cardiovascular events in patients with statin intolerance. Am Heart J. 2021 May; 235: 104-112) All patients provided written informed consent. Patients had to report statin intolerance due to an adverse effect that started or increased during statin therapy and resolved or improved after statin therapy was discontinued. Patients could be enrolled if they tolerated a very low average daily statin dose (e.g., an average daily dose of rosuvastatin <5 mg, atorvastatin <10 mg, simvastatin <10 mg, lovastatin <20 mg, pravastatin <40 mg, fluvastatin <40 mg, or pitavastatin <2 mg). Other lipid-lowering therapies were permitted, including ezetimibe, niacin, bile acid resins, fibrates, and/or proprotein convertase subtilisin kexin type 9 (PCSK9) inhibitors. Full inclusion and exclusion criteria are provided in Nicholls et al. (Am Heart J. 2021).

### Randomization and Trial Regimen

Eligible patients entered a 4-week run-in period during which patients were treated with single-blind placebo. If patients were intolerant to placebo treatment or if adherence was <80% by tablet count, they were not eligible for randomization. Patients who successfully completed the run-in period were randomly assigned in a 1:1 ratio to receive bempedoic acid at an oral dose of 180 mg or matching placebo, administered daily. Beginning 6 months after enrollment, the central laboratory notified the investigator if the patient's LDL-cholesterol level was ≥25% higher than baseline. These patients were counseled on healthy dietary guidelines and reminded to take all lipid-regulating medications. If repeat testing confirmed that LDL-cholesterol value met the threshold criteria, the provider could adjust the lipid-lowering treatment regimen per standard of care and local guidelines.

### Trial End Points

The primary end point was the first occurrence of a composite of death from cardiovascular causes, nonfatal myocardial infarction (MI), nonfatal stroke, or coronary revascularization. The first key secondary end point was time to the first occurrence of the composite of cardiovascular death, nonfatal stroke or MI. Further key secondary end points included time to first occurrence of each of the following outcomes tested in hierarchical order, fatal or nonfatal MI, coronary revascularization, fatal or nonfatal stroke, death from cardiovascular causes, and all-cause mortality.

### Statistical Analysis

This was an event-driven trial designed to provide at least 90% power to detect a 15% relative risk reduction for the primary composite end point at an overall two-sided significance level of 0.05. The trial required a minimum of 1620 primary composite events with >24 months of follow-up for all patients and at least 810 first key secondary end points. The average treatment duration was estimated to be 42 months and a lost-to-follow-up rate of 1% per year. Assuming a 3.59% annual event rate in the placebo group, enrollment of 12,600 patients was required, subsequently amended to 14,000. There were no interim efficacy analyses. A hierarchical approach was prespecified to evaluate sequentially each of the six key secondary efficacy end points with statistical significance at each step required to test the next hypothesis, thereby preserving the study-wise Type I error rate at 5%. All analyses were based on the intent-to-treat principle. The primary efficacy end point was analyzed using Cox proportional hazards model with treatment as a factor to generate the hazard ratio (HR) and 95% confidence interval (CI). The p-value was obtained from a log-rank test.

### (2) Results

### Randomization, Patient Characteristics and Follow-Up

13970 patients were randomized, 6992 assigned to the bempedoic acid group and 6978 to the placebo group. Baseline characteristics of treatment groups were similar (Table 1). Mean (±SD) age was 65.5±9.0 years, 48.2% of patients were female, 45.6% had diabetes, 69.9% had experienced a prior cardiovascular event, 22.7% were taking a statin and 11.5% taking ezetimibe. Mean LDL-cholesterol was 139.0 mg/dL, mean HDL-cholesterol 49.5 mg/dL, and median triglycerides 159.0 mg/dL. Patients were followed for a median of 40.6 months. Premature discontinuation of study drug occurred in 29.1% of bempedoic acid-treated patients and 31.7% of placebo-treated patients. Complete assessment for the primary end point was available for 95.3% of patients and vital status available for 99.4% of patients.

TABLE 1
Demographic and Baseline Characteristics of Patients

| | Bempedoic Acid | | Placebo | |
|---|---|---|---|---|
| Characteristic | (N = 6992) | | (N = 6978) | |
| Age (yrs) | 65.5 ± | 9.1 | 65.5 ± | 8.9 |
| Age, n (%) | | | | |
| <65 yrs. | 2859 | (40.9) | 2907 | (41.7) |
| ≥65 to <75 yrs | 3070 | (43.9) | 3027 | (43.4) |
| ≥75 yrs | 1063 | (15.2) | 1044 | (15.0) |
| Females, n (%) | 3361 | (48.1) | 3379 | (48.4) |
| Race, n (%) | | | | |
| White | 6397 | (91.5) | 6335 | (90.8) |
| Black or African American | 156 | (2.2) | 172 | (2.5) |
| Asian | 130 | (1.9) | 136 | (1.9) |
| American/Mexican Indian or | 240 | (3.4) | 247 | (3.5) |
| Alaska Native | | | | |
| Native Hawaiian or Other | 20 | (0.3) | 17 | (0.2) |
| Pacific Islander | | | | |
| Other | 1 | (<0.1) | 1 | (<0.1) |
| Multiple | 48 | (0.7) | 70 | (1.0) |
| Ethnicity, n (%) | | | | |
| Hispanic or Latino | 1190 | (17.0) | 1143 | (16.4) |
| Body-mass index, kg/m² | 29.9 ± | 5.2 | 30.0 ± | 5.2 |
| LDL-Cholesterol mg/dL, | 139.0 | (34.9) | 139.0 | (35.2) |
| mean (SD) | | | | |
| LDL-Cholesterol category, n (%) | | | | |
| <130 mg/dL | 3074 | (44.0) | 3089 | (44.3) |
| ≥130 mg/dL to <160 mg/dL | 2213 | (31.7) | 2250 | (32.2) |
| ≥160 mg/dL | 1705 | (24.4) | 1639 | (23.5) |
| hsCRP mg/L, median (IQR) | 2.3 | | 2.3 | |
| | (1.2 to 4.5) | | (1.2 to 4.5) | |
| hsCRP category, n (%) | | | | |
| <2 mg/dL | 3070 | (43.9) | 3071 | (44.0) |
| ≥2 mg/dL | 3847 | (55.0) | 3840 | (55.0) |
| Missing | 75 | (1.1) | 67 | (1.0) |
| HDL-Cholesterol mg/dL, | 49.6 ± | 13.3 | 49.4 ± | 13.3 |
| mean (SD) | | | | |
| Non-HDL-Cholesterol mg/dL, | 173.8 ± | 39.5 | 173.9 ± | 40.2 |
| mean (SD) | | | | |
| Total Cholesterol mg/dL, | 223.5 ± | 40.6 | 223.3 ± | 41.1 |
| mean (SD) | | | | |
| Triglycerides mg/dL, | 159.5 | | 158.5 | |
| median (IQR) | (118.0-216.5) | | (118.0-215.0) | |
| Baseline eGFR | | | | |
| mL/min/1.73 m², n (%) | | | | |
| ≥90 | 1216 | (17.4) | 1233 | (17.7) |
| ≥60 to <90 | 4322 | (61.8) | 4282 | (61.4) |
| ≥30 to <60 | 1437 | (20.6) | 1444 | (20.7) |
| Cardiovascular | | | | |
| risk category, n (%) | | | | |
| Primary prevention | 2100 | (30.0) | 2106 | (30.2) |
| Secondary prevention | 4892 | (70.0) | 4872 | (69.8) |
| Coronary artery disease | 3574 | (51.1) | 3536 | (50.7) |
| Peripheral arterial disease | 794 | (11.4) | 830 | (11.9) |
| Cerebrovascular atherosclerotic | 1027 | (14.7) | 1040 | (14.9) |
| disease | | | | |
| Glycemic status, n (%) | | | | |
| No diabetes | 3848 | (55.0) | 3749 | (53.7) |
| Normoglycemic | 937 | (13.4) | 864 | (12.4) |
| Prediabetes§ | 2911 | (41.6) | 2885 | (41.3) |
| Diabetes^{‡} | 3144 | (45.0) | 3229 | (46.3) |
| Inadequately controlled diabetes† | 1356 | (19.4) | 1369 | (19.6) |
| Baseline statin use, n (%) | 1601 | (22.9) | 1573 | (22.5) |
| Baseline ezetimibe use, n (%) | 803 | (11.5) | 809 | (11.6) |

*To convert the values for cholesterol to millimoles per liter, multiply by 0.02586. To convert the values for triglycerides to millimoles per liter, multiply by 0.01129. LDL-C denotes low density lipoprotein cholesterol, hsCRP high sensitivity C-reactive protein, HDL-C high density lipoprotein cholesterol.
§Patients at baeline with no medical history of type 2 diabetes, no prior use of glucose-lowering medication, and a HbA1C measurement of 5.7% to 6.4%, or 1 or more measurements of fasting glucose of 100 mg/dL (5.6 mmol/L) to 125 mg/dL (6.9 mmol/L), but not more than 1 value of fasting glucose ≥126 mg/dl (7.0 mmol/L).
^{‡}At baseline, medical history of type 2 diabetes, prior glucose-lowering medication, HbA1C measurement >6.5%, or two or more measurements of fasting glucose ≥126 mg/dl (7.0 mmol/L).
†Patients with type 2 diabetes and an HbA1C of 7.0% or greater at baseline.

### Effect on LDL-Cholesterol and High-Sensitivity C-Reactive Protein

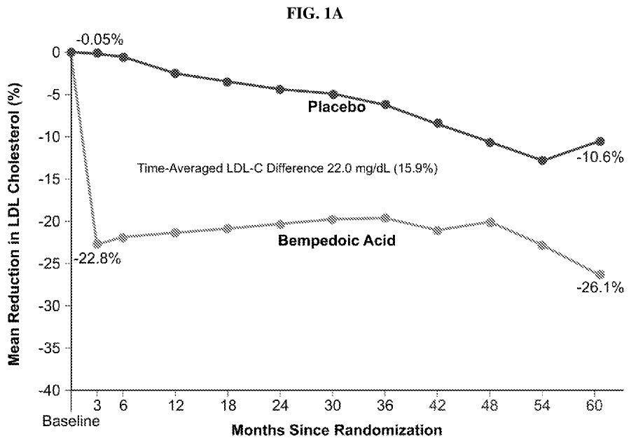

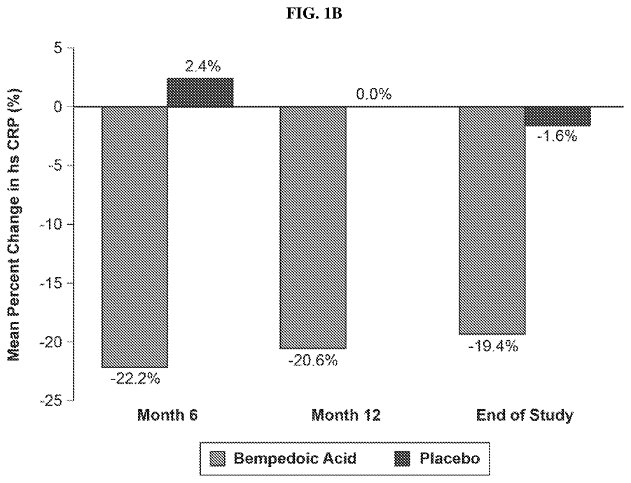

FIG. 1A shows the effect of the trial regimens over time on LDL-cholesterol and Panel B shows the effect on high-sensitivity C-reactive protein (hsCRP). From a baseline mean LDL-cholesterol of 139.0 mg/dL, placebo-subtracted reduction in the bempedoic acid group after 6 months of treatment was 29.2 mg/dL (21.1%) (95% confidence interval [CI]−21.9 to −20.3%) and time-averaged reduction 22.0 mg/dL (15.9%). Among placebo patients, 15.6% crossed over to additional lipid-lowering therapy, compared with 9.4% of bempedoic acid patients. From a median baseline value of 2.3 mg/L, after 6 months treatment, bempedoic acid reduced hsCRP by 21.6% (95% CI −23.7% to −19.6%). Effects of trial regimens on tertiary lipid biomarkers are provided in Table 2.

TABLE 2
Selected Tertiary Lipid Endpoints

| | Bempedoic | | Placebo | |
|---|---|---|---|---|
| | Acid | | | |
| | (N = 6992) | | (N = 6978) | |
| | Observed mean ± | | | |
| Lipid or Biomarker | SD or median (IQR) | | | |
| Percent change from baseline | −17.2% ± | 21.2 | −0.4% ± | 21.4 |
| to Month 6 in non-HDL-C | | | | |
| Percent change from baseline | −17.1% ± | 22.0 | −2.0% ± | 22.3 |
| to Month 12 in non-HDL-C | | | | |
| Percent change from baseline | −16.2% ± | 24.3 | −3.8% ± | 24.5 |
| to Month 24 in non-HDL-C | | | | |
| Percent change from baseline | −16.9% ± | 16.7 | −0.57% ± | 16.3 |
| to Month 6 in total cholesterol | | | | |
| Percent change from baseline | −14.8% ± | 17.3 | −1.8% ± | 17.1 |
| to Month 12 in total cholesterol | | | | |
| Percent change from baseline | −5.8% ± | 18.0 | 0.66% ± | 15.1 |
| to Month 6 in HDL-C | | | | |
| Percent change from baseline | −5.7% ± | 18.9 | 0.70% ± | 16.2 |
| to Month 12 in HDL-C | | | | |
| Percent change from baseline | −3.4% | | −1.7% | |
| to Month 6 in triglycerides | (−23.7% to | | (−19.5% to | |
| | 22.1%) | | 19.5%) | |
| Percent change from baseline | −4.8% | | −2.3% | |
| to Month 12 in triglycerides | (−24.8% to | | (−20.8% to | |
| | 21.6%) | | 20.4%) | |
| Change from baseline to | | | | |
| Month 6 in HbA1c (%) | | | | |
| All patients | −0.003 ± | 0.6 | 0.05 ± | 0.7 |
| Inadequately Controlled | −0.20 ± | 1.0 | −0.14 ± | 1.2 |
| Diabetes at Baseline | | | | |
| Change from baseline to | | | | |
| Month 12 in HbA1c (%) | | | | |
| All patients | 0.04 ± | 0.74 | 0.06 ± | 0.7 |
| Inadequately Controlled | −0.06 ± | 1.3 | −0.03 ± | 1.2 |
| Diabetes at Baseline | | | | |
| Change from baseline | | | | |
| to Month 6 in fasting | | | | |
| glucose (mg/dL) | | | | |
| All patients | −1.0 | | 0.5 | |
| | (−9.0 to 7.0) | | (−7.5 to 8.5) | |
| Inadequately Controlled | −4.0 | | 0.5 | |
| Diabetes at Baseline | (−32.0 to 25.0) | | (−26.5 to 29.0) | |
| Change from baseline | | | | |
| to Month 12 in fasting | | | | |
| glucose (mg/dL) | | | | |

| | | |
|---|---|---|
| All patients | 0.0 | 1.0 |
| | (−8.0 to 8.5) | (−7.0 to 10.0) |
| Inadequately Controlled | −2.75 | 0.5 |
| Diabetes at Baseline | (−32.5 to 30.5) | (−28.3 to 29.0) |

### Efficacy Endpoints

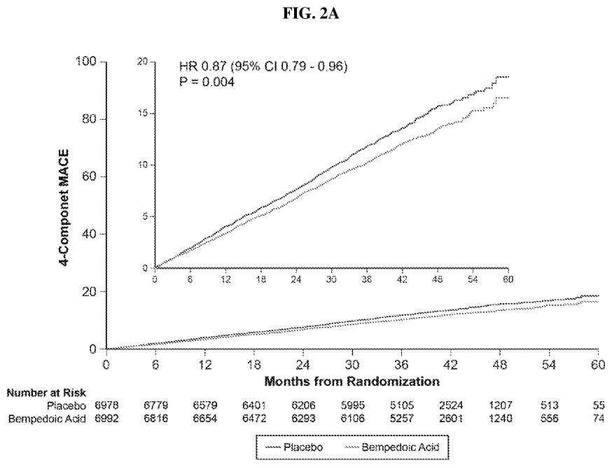

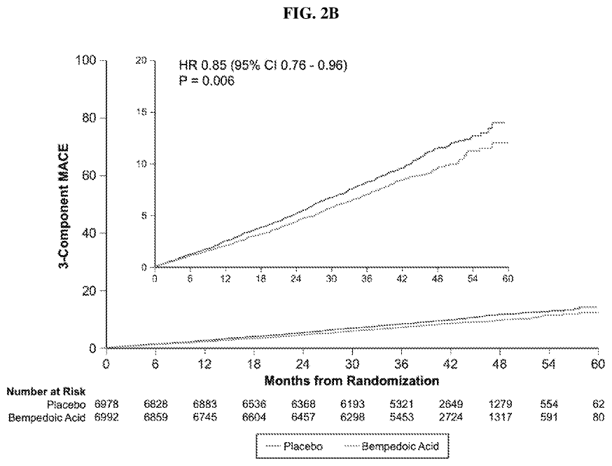

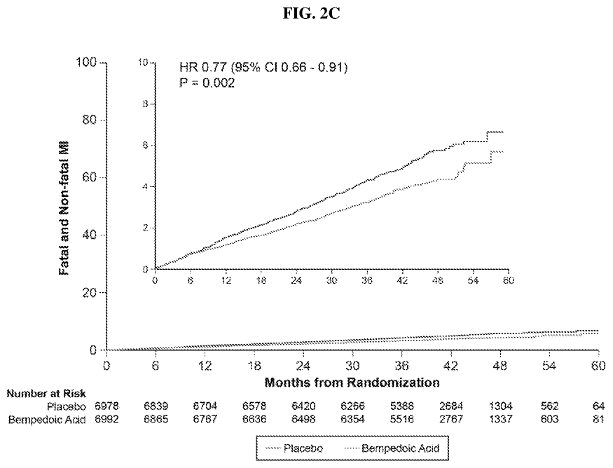

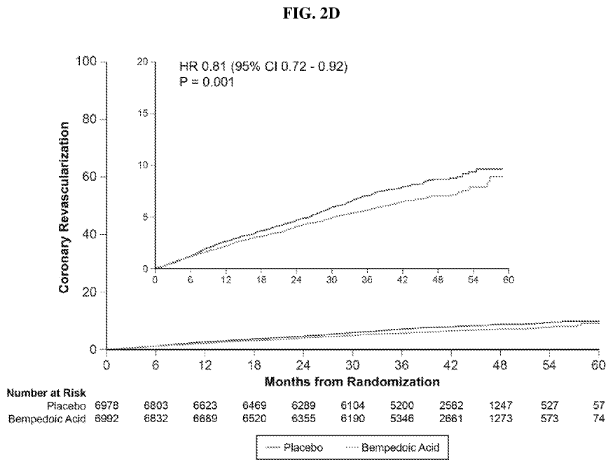

The primary composite end point occurred in 819 patients (11.7%) in the bempedoic acid group and 927 patients (13.3%) in the placebo group HR, 0.87 (95% CI 0.79 to 0.96), P=0.004. (Table 3 and FIG. 2A) Bempedoic acid significantly reduced the risk of the first key secondary composite end point, time to death from cardiovascular causes, nonfatal MI or stroke, which occurred in 575 patients (8.2%) in the bempedoic acid group and in 663 patients (9.5%) in the placebo group, HR 0.85, 95% CI 0.76 to 0.96, P=0.006. (Table 3 and FIG. 2B) The second key secondary end point, time to first occurrence of fatal or nonfatal MI occurred in 261 (3.7%) of patients in the bempedoic acid treatment group and 334 (4.8%) of patients in the placebo group, HR 0.77 (95% CI 0.66 to 0.91), P=0.002. (Table 3 and FIG. 2C) The third key secondary end point, time to coronary revascularization, occurred in 435 (6.2%) patients in the bempedoic acid group compared with 529 (7.6%) patients in the placebo group, HR 0.81, 95% CI 0.72 to 0.92, P=0.001. (Table 3 and FIG. 2D) Compared with placebo, there were no significant effects of bempedoic acid on the other key secondary end points, time to first occurrence of stroke, death from cardiovascular causes, and all-cause mortality. (Table 3).

Results for the primary end point in prespecified subgroups were determined. There was no statistically significant heterogeneity for any subgroup except for a lower HR in the primary compared with the secondary prevention population, HR 0.68 vs. 0.91 (interaction P=0.03).

TABLE 3
Time to Event Efficacy End Points for the Bempedoic Acid
Treatment Group Compared with Placebo

| | Bempedoic | | | |
|---|---|---|---|---|
| | Acid | Placebo | | |
| | (N = 6992) | (N = 6978) | Hazard Ratio | P |
| Outcome | number of patients (%) | | (95% CI) | Value |
| Primary efficacy | 819 (11.7) | 927 (13.3) | 0.87 (0.79-0.96) | 0.004 |
| endpoint | | | | |
| (MACE-4)* | | | | |

Key secondary efficacy endpoints

| | | | | |
|---|---|---|---|---|
| 3-component | 575 (8.2) | 663 (9.5) | 0.85 (0.76-0.96) | 0.006 |
| MACE^{§} | | | | |
| Fatal and non- | 261 (3.7) | 334 (4.8) | 0.77 (0.66-0.91) | 0.002 |
| fatal MI | | | | |
| Coronary | 435 (6.2) | 529 (7.6) | 0.81 (0.72-0.92 | 0.001 |
| revascularization | | | | |
| Fatal and non-fatal | 135 (1.9) | 158 (2.3) | 0.85 (0.67-1.07) | NA† |
| stroke† | | | | |
| Death from cardio- | 269 (3.8) | 257 (3.7) | 1.04 (0.88-1.24) | NA† |
| vascular causes^{†} | | | | |
| All-cause mortality^{†} | 434 (6.2) | 420 (6.0) | 1.03 (0.90-1.18) | NA† |

Additional secondary time-to-event end points

| | | | | |
|---|---|---|---|---|
| All-cause mortality, | 962 (13.8) | 1062 (15.2) | 0.89 (0.82-0.97) | NA† |
| nonfatal MI, nonfatal | | | | |
| stroke, or coronary | | | | |
| revascularization | | | | |
| 5 component MACE^{‡} | 831 (11.9) | 952 (13.6) | 0.86 (0.78-0.94) | NA† |
| Non-fatal MI | 236 (3.4) | 317 (4.5) | 0.73 (0.62-0.87 | NA† |
| Fatal MI | 29 (0.4) | 21 (0.3) | 1.38 (0.79-2.42) | NA† |
| Non-fatal stroke | 119 (1.7) | 144 (2.1) | 0.82 (0.64-1.05) | NA† |
| Fatal stroke | 18 (0.3) | 16 (0.2) | 1.12 (0.57-2.20) | NA† |
| Fatal and nonfatal | 118 (1.7) | 150 (2.1) | 0.78 (0.61-0.99) | NA† |
| non-hemorrhagic | | | | |
| stroke | | | | |
| Fatal and nonfatal | 20 (0.3) | 9 (0.1) | 2.21 (1.01-4.85) | NA† |
| hemorrhagic stroke | | | | |
| Hospitalization for | 91 (1.3) | 137 (2.0) | 0.66 (0.50-0.86) | NA† |
| unstable angina | | | | |
| New-onset type 2 | 429/3848 | 433/3749 | 0.95 (0.83-1.09) | NA† |
| diabetes mellitus^{#} | (11.1) | (11.5) | | |

Change in secondary lipid and biomarker efficacy end points

| | Mean or Median | | Difference | |
|---|---|---|---|---|
| | (95% CI) | | (95% CI) | |
| Mean change from | −21.1% | −0.8% | −20.3% | |
| baseline to month 6 | (−21.6 to | (−1.4% to | (−21.1% to | NA† |
| in mean LDL-C^{€} | −20.5%) | −0.2%) | −19.5%) | |
| Median change from | −22.2% | 2.4% | −21.6% | NA† |
| baseline to month 6 | (−23.5 to | (0 to | (−23.7 to | |
| in hsCRP | −20.8%) | 4.2%) | −19.6%) | |
| Change from base- | −0.04% | −0.01% | −0.03% | NA† |
| line to month 12 | (−0.12 to | (−0.09 to | (−0.14 to | |
| in HbA1C^{€} in | 0.03) | 0.06) | 0.08) | |
| patients with | | | | |
| inadequately | | | | |
| controlled type | | | | |
| 2 diabetes mellitus^{£} | | | | |

MACE denotes major adverse cardiovascular events,
MI myocardial infarction,
CI confidence intervals,
LDL-C low density lipoprotein cholesterol,
hsCRP high-sensitivity C-reactive protein.
†As prespecified in the hierarchical testing procedure, all p values after the first nonsignificant p value are not presented.
*The primary efficacy endpoint (MACE-4) is the time to first occurrence of an adjudicated event for a composite that includes death from cardiovascular causes, nonfatal MI, nonfatal stroke, or coronary revascularization.
^{§}Time to first occurrence of the composite endpoint of death from cardiovascular causes, nonfatal MI, or nonfatal stroke (MACE-3).
^{‡}Time to first occurrence of death from cardiovascular causes, nonfatal MI, nonfatal stroke, coronary revascularization, or hospitalization for unstable angina (MACE-5).
^{#}Glycated hemoglobin level >6.5% or two or more measurements of fasting glucose ≥126 mg/dl (7.0 mmol/L) in patients with baseline glycemic status of no diabetes.
^{€}Results adjusted for baseline LDL-C or HbA1c values, based on a pattern-mixed model for missing data.
^{£}Defined as diabetes and a glycated hemoglobin level of 7% or greater at baseline.

### Adverse Effects

Adverse events are reported in Table 4. There were not clinically meaningful between group differences in overall rates of adverse events, serious adverse events, or adverse events leading to drug discontinuation. Investigator-reported prespecified adverse events of special interest were balanced except for more frequent hepatic enzyme elevations (4.5% vs 3.0%) and renal impairment (11.5% vs. 8.6%) in the bempedoic acid group. Musculoskeletal adverse events occurred in 15.0% of bempedoic acid treated patients and 15.4% of placebo patients. Rates of liver transaminase elevations >3×ULN were more frequent in the bempedoic acid group. Mean changes in creatinine and uric acid from baseline were larger for bempedoic acid compared with placebo. A higher incidence of hyperuricemia (10.9% vs 5.6%), gout (3.1% vs 2.1%) and cholelithiasis (2.2% vs 1.2%) occurred in the bempedoic acid group.

TABLE 4
Adverse Events and Safety Laboratory Findings

| | Bempedoic | | | |
|---|---|---|---|---|
| | Acid | | Placebo | |
| | (N = 7001), | | (N = 6964), | |
| | n (%) | | n (%) | |
| Any treatment emergent | 6041 | (86.3) | 5921 | (85.0) |
| adverse event | | | | |
| Serious treatment emergent | 1778 | (25.4) | 1743 | (25.0) |
| adverse event | | | | |
| Adverse event leading to drug | 768 | (11.0) | 726 | (10.4) |
| discontinuation | | | | |
| Prespecified Adverse | | | | |
| Events of Special Interest | | | | |
| Any muscle disorder | 1052 | (15.0) | 1070 | (15.4) |
| Myalgia | 393 | (5.6) | 471 | (6.8) |
| Discontinuation of treatment due | 124 | (1.8) | 129 | (1.9) |
| to myalgia | | | | |
| New onset diabetes in patients | 621/3856 | (16.1) | 640/3740 | (17.1) |
| without diabetes at baseline | | | | |
| New onset diabetes in patients | 569/2918 | (19.5) | 586/2877 | (20.4) |
| with pre-diabetes at baseline^{#} | | | | |
| New onset diabetes in patients | 52/938 | (5.5) | 54/863 | (6.3) |
| with normoglycemia at baseline^{#} | | | | |
| Worsening hyperglycemia* | 713/3145 | (22.7) | 746/3224 | (23.1) |
| Hypoglycemia | 304 | (4.3) | 267 | (3.8) |
| Metabolic acidosis | 13 | (0.2) | 11 | (0.2) |
| Elevated hepatic enzymes | 317 | (4.5) | 209 | (3.0) |
| Renal impairment | 802 | (11.5) | 599 | (8.6) |
| Neurocognitive disorders | 58 | (0.8) | 69 | (1.0) |
| Atrial fibrillation | 229 | (3.3) | 246 | (3.5) |
| Adjudicated tendon rupture | 86 | (1.2) | 66 | (0.9) |
| Tendinopathies | 118 | (1.7) | 128 | (1.8) |
| Malignancies | 329 | (4.7) | 345 | (5.0) |
| Other Adverse Events | | | | |
| Hyperuricemia | 763 | (10.9) | 393 | (5.6) |
| Gout | 215 | (3.1) | 143 | (2.1) |
| Cholelithiasis | 152 | (2.2) | 81 | (1.2) |
| Laboratory Results | | | | |
| after 6 months, mg/dL | | | | |
| Change from baseline in | 0.76 ± | 1.2 | −0.03 ± | 1.0 |
| uric acid levels | | | | |
| Change from baseline in | 0.05 ± | 0.2 | 0.01 ± | 0.2 |
| creatinine levels | | | | |
| Laboratory Results | | | | |
| after 12 months | | | | |
| Change from baseline in glycated | 0.04 ± | 0.74 | 0.06 ± | 0.70 |
| hemoglobin level (%)^{@} | | | | |
| Enzyme abnormalities at any visit | | | | |
| Creatine kinase levels >5x ULN | 45 | (0.6) | 40 | (0.6) |
| (single occurrence) | | | | |
| Creatine kinase levels >5x ULN | 8 | (0.1) | 8 | (0.1) |
| (repeated and confirmed) | | | | |
| Alanine aminotransferase level | 83 | (1.2) | 53 | (0.8) |
| >3 times ULN^{&} | | | | |
| Aspartate aminotransferase level | 80 | (1.1) | 43 | (0.6) |
| >3 times ULN^{&} | | | | |

ULN denotes upper limit of normal
*In patients with diabetes at baseline
^{#}Patients with prediabetes at baseline were defined as: No past medical history of diabetes and with glycated hemoglobin level of ≥5.7% and <6.5% or 1 or more measurement of fasting glucose ≥100 mg/dL (5.6 mmol/L), but not more than 1 value of fasting glucose ≥126 mg/dl (7.0 mmol/L). Patients with normoglycemia at baseline did not meet criteria for prediabetes.
^{@}Not a prespecified safety measure
^{&}Repeated and confirmed

### Discussion

In primary and secondary prevention patients unable or unwilling to take guideline-recommended doses of statins, treatment with bempedoic acid resulted in a significant 13% reduction in the primary composite end point of death from cardiovascular causes, stroke, MI, or coronary revascularization. Hierarchical testing of the first three key secondary end points also showed significant benefits for bempedoic acid treatment. The composite of death from cardiovascular causes, stroke or MI was reduced by 15%, the incidence of fatal or nonfatal MI reduced by 23%, and coronary revascularization reduced by 19%. After 6 months treatment, bempedoic acid, compared with placebo, reduced LDL-cholesterol by a mean of 29.2 mg/dL (21.1%) and hsCRP by a median of 21.6%.

Bempedoic acid treatment was well tolerated with rates of discontinuation for any reason, including musculoskeletal complaints, similar to placebo (Table 4). There were no differences for other prespecified adverse events of special interest, including new onset or worsening of diabetes mellitus, hypoglycemia and metabolic acidosis, neurocognitive disorders, atrial fibrillation, tendinopathies including tendon rupture and malignancies. A higher incidence of cholelithiasis was reported.

The similar incidence of muscle-related complaints in the bempedoic acid and placebo groups confirms the utility of bempedoic acid as an alternative LDL-cholesterol lowering therapy in patients unable or unwilling to take statins (Table 4).

The time-averaged reduction in LDL-cholesterol of 22.0 mg/dL (0.57 mmol/L) would be expected to result in approximately the reduction observed in cardiovascular events (FIG. 1A).

In conclusion, in primary and secondary prevention patients unable to tolerate recommended doses of statins, bempedoic acid lowered LDL-cholesterol and hsCRP and significantly reduced the primary composite end point, time to death from cardiovascular causes, nonfatal MI, nonfatal stroke, or coronary revascularization by 13%. The narrower key secondary end point that excluded revascularization was reduced by 15% and the incidence of fatal or nonfatal MI was reduced by 23%.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

We claim:

1. A method of reducing the risk of myocardial infarction in an adult with established cardiovascular disease or two or more risk factors for cardiovascular disease, the method comprising orally administering daily to the adult a pharmaceutical formulation comprising 180 mg bempedoic acid; and one or more pharmaceutically acceptable excipients selected from the group consisting of colloidal silicon dioxide, a hydroxyl propyl cellulose, lactose monohydrate, magnesium stearate, microcrystalline cellulose, sodium starch glycolate, a povidone, and sodium lauryl sulfate, and combinations thereof.

2. The method of claim 1, wherein the adult has established cardiovascular disease.

3. The method of claim 1, wherein the adult has two or more risk factors for cardiovascular disease.

4. The method of claim 3, wherein the two or more risk factors for cardiovascular disease are selected from the group consisting of smoking, diabetes mellitus (DM) hypertension, high body mass index (BMI), obesity, and dyslipidemia.

5. The method of claim 1, wherein the adult has primary hyperlipidemia.

6. The method of claim 5, wherein the primary hyperlipidemia is heterozygous familial hypercholesterolemia.

7. The method of claim 1, wherein the pharmaceutical formulation comprises colloidal silicon dioxide; hydroxyl propyl cellulose; lactose monohydrate; magnesium stearate; microcrystalline cellulose; and sodium starch glycolate.

8. The method of claim 1, wherein the pharmaceutical formulation comprises colloidal silicon dioxide; a hydroxy propyl cellulose; lactose monohydrate; magnesium stearate; microcrystalline cellulose; a povidone, sodium lauryl sulfate, and sodium starch glycolate.

9. The method of claim 1, further comprising administering to the adult another low-density lipoprotein cholesterol (LDL-C) lowering therapy.

10. The method of claim 9, wherein the other LDL-C-lowering therapy comprises niacin, a bile acid resin, a fibrate, or a proprotein convertase subtilisin kexin type 9 (PCSK9) inhibitor.

11. The method of claim 9, wherein the other LDL-C-lowering therapy comprises a statin.

12. The method of claim 11, wherein the statin is selected from the group consisting of atorvastatin, fluvastatin, lovastatin, pitavastatin, pravastatin, rosuvastatin, simvastatin, and combinations thereof.

13. The method of claim 9, wherein the other LDL-C-lowering therapy comprises ezetimibe.

14. The method of claim 1, wherein the pharmaceutical formulation further comprises 10 mg ezetimibe.

15. The method of claim 1, wherein the adult is statin intolerant.
